SEQUENCE LISTING (27 sequences)

SEQ ID NO: 1 - DNA, 1770 bp, S. cerevisiae
  CDS (1)...(1770)
atgagtggagtatcaaataaaacagtatcaattaatggttggtatggcatgccaattcatttactaagggaagaaggcgactttgcccagtttatgattctaaccatcaacgaattaaaaatagccatacatggttacctcagaaataccccatggtacaacatgttgaaggattatttgtttgtgatcttttgttacaagctaataagtaattttttttatctgttgaaagtttatgggccggtgaggttagcagtgagaacatacgagcatagttccagaagattgtttcgttggttattggactcaccatttttgaggggtaccgtagaaaaggaagtcacaaaggtcaaacaatcgatcgaagacgaactaattagatcggactctcagttaatgaatttcccacagttgccatccaatgggatacctcaggatgatgttattgaagagctaaataaattgaacgacttgataccacatacccaatggaaggaaggaaaggtctctggtgccgtttaccacggtggtgatgatttgatccacttacaaacaatcgcatacgaaaaatattgcgttgccaatcaattacatcccgatgtctttcctgccgtacgtaaaatggaatccgaagtggtttctatggttttaagaatgtttaatgccccttctgatacaggttgtggtaccacaacttcaggtggtacagaatccttgcttttagcatgtctgagcgctaaaatgtatgcccttcatcatcgtggaatcaccgaaccagaaataattgctcccgtaactgcacatgctgggtttgacaaagctgcttattactttggcatgaagctacgccacgtggagctagatccaacgacatatcaagtggacctgggaaaagtgaaaaaattcatcaataagaacacaattttactggtcggttccgctccaaactttcctcatggtattgccgatgatattgaaggattgggtaaaatagcacaaaaatataaacttcctttacacgtcgacagttgtctaggttcctttattgtttcatttatggaaaaggctggttacaaaaatctgccattacttgactttagagtcccgggagtcacctcaatatcatgtgacactcataaatatggatttgcaccaaaaggctcgtcagttataatgtatagaaacagcgacttacgaatgcatcagtattacgtaaatcctgcttggactggcgggttatatggctctcctacattagcagggtccaggcctggtgctattgtcgtaggttgttgggccactatggtcaacatgggtgaaaatgggtacattgagtcgtgccaagaaatagtcggtgcagcaatgaagtttaaaaaatacatccaggaaaacattccagacctgaatataatgggcaaccctagatattcagtcatttcattttcttcaaagaccttgaacatacacgaactatctgacaggttgtccaagaaaggctggcatttcaatgccctacaaaagccggttgcactacacatggccttcacgagattgagcgctcatgttgtggatgagatctgcgacattttacgtactaccgtgcaagagttgaagagcgaatcaaattctaaaccatccccagacggaactagcgctctatatggtgtcgccgggagcgttaaaactgctggcgttgcagacaaattgattgtgggattcctagacgcattatacaagttgggtccaggagaggataccgccaccaagtag

SEQ ID NO: 2 - protein, 589 aa, S. cerevisiae
MSGVSNKTVSINGWYGMPIHLLREEGDFAQFMILTINELKIAIHGYLRNTPWYNMLKDYLFVIFCYKLISNFFYLLKVYGPVRLAVRTYEHSSRRLFRWLLDSPFLRGTVEKEVTKVKQSIEDELIRSDSQLMNFPQLPSNGIPQDDVIEELNKLNDLIPHTQWKEGKVSGAVYHGGDDLIHLQTIAYEKYCVANQLHPDVFPAVRKMESEVVSMVLRMFNAPSDTGCGTTTSGGTESLLLACLSAKMYALHHRGITEPEIIAPVTAHAGFDKAAYYFGMKLRHVELDPTTYQVDLGKVKKFINKNTILLVGSAPNFPHGIADDIEGLGKIAQKYKLPLHVDSCLGSFIVSFMEKAGYKNLPLLDFRVPGVTSISCDTHKYGFAPKGSSVIMYRNSDLRMHQYYVNPAWTGGLYGSPTLAGSRPGAIVVGCWATMVNMGENGYIESCQEIVGAAMKFKKYIQENIPDLNIMGNPRYSVISFSSKTLNIHELSDRLSKKGWHFNALQKPVALHMAFTRLSAHVVDEICDILRTTVQELKSESNSKPSPDGTSALYGVAGSVKTAGVADKLIVGFLDALYKLGPGEDTATK

SEQ ID NO: 3 - DNA, 1629 bp, C. elegans
  CDS (1)...(1629)
atggattttgcactggagcaatatcatagtgcaaaggatttgttaatatttgagcttcgaaagttcaatccaattgttctggtttctagtactattgttgcaacatacgtactcaccaatctgagacatatgcatttagatgaaatgggcatccggaaacgtttgagcacttggtttttcaccactgtaaagcgtgtgcctttcatcaggaaaatgattgacaaacaactaaacgaagtaaaggacgagcttgagaaaagtctgagaattgtggatcgaagcaccgaatacttcactacaatcccaagccattcagttggaagaactgaagtacttcgccttgctgccatctatgatgatttggaaggaccagcttttttggaaggaagagtatctggagcagtcttcaatagagaagacgacaaggacgaacgggagatgtatgaggaggtgttcggaaaatttgcctggaccaacccactttggccaaaattgttccctggagtgagaatcatggaggctgaagttgttcgcatgtgttgtaatatgatgaatggagattcggagacatgtggaactatgtcaactggtggatccatttcaattcttttggcgtgcctggctcatcgtaatcgtcttttgaaaagaggagaaaagtacacagagatgattgtcccatcatccgtccatgcagcgttcttcaaagctgccgaatgtttccgtatcaaagttcgcaagattccagttgatcctgttactttcaaagtagaccttgtcaaaatgaaagccgcaattaacaagagaacatgtatgttagttggatctgctccaaactttccatttggaactgttgatgacattgaagctattggacagctaggacttgaatatgacatcccagttcatgttgatgcttgtcttggtggtttccttcttccattccttgaagaagacgagattcgctatgacttccgtgttcctggtgtatcttcgatttctgcagatagtcacaaatacggactcgctccaaaggggtcatcagttgttctttatcgcaataaggaacttcttcataatcagtacttctgtgatgctgattggcaaggaggtatctatgcatcggctactatggaaggatcacgcgctgggcacaacattgcactttgctgggccgcaatgctttatcacgctcaggaaggatacaaggccaatgctagaaagattgttgacactacaagaaagattagaaatggactttcaaacattaagggaatcaaattacaagggccaagtgatgtttgtattgttagctggacaaccaatgatggagttgaactctacagattccataacttcatgaaggaaaaacattggcaactgaatggacttcaattcccagctggagttcatatcatggtcactatgaatcatactcatcctggactcgctgaagctttcgtcgccgattgcagagctgcagttgagtttgtcaaaagccacaaaccatcggaatccgacaagacaagtgaagcagccatctacggacttgctcaaagtattccagaccgatcgcttgttcacgagtttgctcacagctatatcgatgctgtttatgctttaacagagtga

SEQ ID NO: 4 - protein, 542 aa, C. elegans
MDFALEQYHSAKDLLIFELRKFNPIVLVSSTIVATYVLTNLRHMHLDEMGIRKRLSTWFFTTVKRVPFIRKMIDKQLNEVKDELEKSLRIVDRSTEYFTTIPSHSVGRTEVLRLAAIYDDLEGPAFLEGRVSGAVFNREDDKDEREMYEEVFGKFAWTNPLWPKLFPGVRIMEAEVVRMCCNMMNGDSETCGTMSTGGSISILLACLAHRNRLLKRGEKYTEMIVPSSVHAAFFKAAECFRIKVRKIPVDPVTFKVDLVKMKAAINKRTCMLVGSAPNFPFGTVDDIEAIGQLGLEYDIPVHVDACLGGFLLPFLEEDEIRYDFRVPGVSSISADSHKYGLAPKGSSVVLYRNKELLHNQYFCDADWQGGIYASATMEGSRAGHNIALCWAAMLYHAQEGYKANARKIVDTTRKIRNGLSNIKGIKLQGPSDVCIVSWTTNDGVELYRFHNFMKEKHWQLNGLQFPAGVHIMVTMNHTHPGLAEAFVADCRAAVEFVKSHKPSESDKTSEAAIYGLAQSIPDRSLVHEFAHSYIDAVYALTE

SEQ ID NO: 5 - DNA, 1707 bp, Mus musculus
  CDS (1)...(1707)
atgcccggaaccgacctcctcaagctgaaggacttcgagccttatttggagattttggaatcttattccacaaaagccaagaattatgtgaatggatattgcaccaaatatgagccctggcagctcattgcgtggagtgtcctgtgtactctgctgatagtctgggtgtatgagcttatcttccagccagagagtttatggtctcggtttaaaaaaaaattatttaagcttatcaggaagatgccatttattggacgtaagatcgaacaacaggtgagcaaagccaagaaggatcttgtcaagaacatgccattcctaaaggtggacaaggattatgtgaaaactctgcctgctcagggtatgggcacagctgaggttctggagagactcaaggagtacagctccatggatggttcctggcaagaagggaaagcctcaggagctgtgtacaatggggaaccgaagctcacggagctgctggtgcaggcttatggagaattcacgtggagcaatccactgcatccagatatcttccctggattgcggaagttagaggcagaaatcgttaggatgacttgttccctcttcaatgggggaccagattcctgtggatgtgtgacttctgggggaacggaaagcatcctgatggcctgcaaagcttaccgggacttggcgttagagaaggggatcaaaactccagaaattgtggctcccgagagtgcccatgctgcattcgacaaagcagctcattattttgggatgaagattgtccgagttgcactgaaaaagaacatggaggtggatgtgcaggcaatgaagagagccatctccaggaacacagctatgctggtctgttctaccccacagtttcctcatggtgtgatggatcctgtccccgaagtggccaagttaactgtcagatataaaatcccactccatgtggatgcttgtctggggggcttcctcattgtcttcatggagaaagcagggtacccactggagaaaccatttgatttccgggtgaaaggtgtgaccagcatttcagcagatactcataagtatggctatgctcctaaaggttcatcagtggtgatgtactctaacgagaagtacaggacgtaccagttctttgttggtgcagactggcaaggtggtgtctacgcatctccaagcatagctggctcacggcctggtggcatcattgcagcctgttgggcggccttgatgcacttcggtgagaacggctatgttgaagctaccaaacagatcatcaaaactgctcgcttcctgaagtcagaactggaaaacatcaaaaacatcttcattttcggtgatcctcaattgtcagttattgctctgggatccaacgattttgacatttaccgactatctaatatgatgtctgctaaggggtggaattttaactacctgcagttcccaagaagcattcatttctgcattacgttagtacatactcggaagcgagtggcgatccagttcctaaaggatatccgggaatcagtcacacaaatcatgaagaatcctaaagctaagaccacaggaatgggtgccatctatggcatggcccaggcaaccattgacaggaagctggttgcagaaatatcctccgtcttcttggactgcctttatactacggaccccgtgactcagggcaaccagatgaacggttctccaaagccccgctga

SEQ ID NO: 6 - protein, 568 aa, Mus musculus
MPGTDLLKLKDFEPYLEILESYSTKAKNYVNGYCTKYEPWQLIAWSVLCTLLIVWVYELIFQPESLWSRFKKKLFKLIRKMPFIGRKIEQQVSKAKKDLVKNMPFLKVDKDYVKTLPAQGMGTAEVLERLKEYSSMDGSWQEGKASGAVYNGEPKLTELLVQAYGEFTWSNPLHPDIFPGLRKLEAEIVRMTCSLFNGGPDSCGCVTSGGTESILMACKAYRDLALEKGIKTPEIVAPESAHAAFDKAAHYFGMKIVRVALKKNMEVDVQAMKRAISRNTAMLVCSTPQFPHGVMDPVPEVAKLTVRYKIPLHVDACLGGFLIVFMEKAGYPLEKPFDFRVKGVTSISADTHKYGYAPKGSSVVMYSNEKYRTYQFFVGADWQGGVYASPSIAGSRPGGIIAACWAALMHFGENGYVEATKQIIKTARFLKSELENIKNIFIFGDPQLSVIALGSNDFDIYRLSNMMSAKGWNFNYLQFPRSIHFCITLVHTRKRVAIQFLKDIRESVTQIMKNPKAKTTGMGAIYGMAQATIDRKLVAEISSVFLDCLYTTDPVTQGNQMNGSPKPR

SEQ ID NO: 7 - DNA, 1707 bp, Homo sapiens
  CDS (1)...(1707)
atgcctagcacagaccttctgatgttgaaggcctttgagccctacttagagattttggaagtatactccacaaaagccaagaattatgtaaatggacattgcaccaagtatgagccctggcagctaattgcatggagtgtcgtgtggaccctgctgatagtctggggatatgagtttgtcttccagccagagagtttatggtcaaggtttaaaaagaaatgttttaagctcaccaggaagatgcccattattggtcgtaagattcaagacaagttgaacaagaccaaggatgatattagcaagaacatgtcattcctgaaagtggacaaagagtatgtgaaagctttaccctcccagggtctgagctcatctgctgttttggagaaacttaaggagtacagctctatggacgccttctggcaagaggggagagcctctggaacagtgtacagtggggaggagaagctcactgagctccttgtgaaggcttatggagattttgcatggagtaaccccctgcatccagatatcttcccaggactacgcaagatagaggcagaaattgtgaggatagcttgttccctgttcaatgggggaccagattcgtgtggatgtgtgacttctgggggaacagaaagcatactcatggcctgcaaagcatgtcgggatctggcctttgagaaggggatcaaaactccagaaattgtggctccccaaagtgcccatgctgcatttaacaaagcagccagttactttgggatgaagattgtgcgggtcccattgacgaagatgatggaggtggatgtgagggcaatgagaagagctatctccaggaacactgccatgctcgtctgttctaccccacagtttcctcatggtgtaatagatcctgtccctgaagtggccaagctggctgtcaaatacaaaataccccttcatgtcgacgcttgtctgggaggcttcctcatcgtctttatggagaaagcaggatacccactggagcacccatttgatttccgggtgaaaggtgtaaccagcatttcagctgacacccataagtatggctatgccccaaaaggctcatcattggtgttgtatagtgacaagaagtacaggaactatcagttcttcgtcgatacagattggcagggtggcatctatgcttccccaaccatcgcaggctcacggcctggtggcattagcgcagcctgttgggctgccttgatgcacttcggtgagaacggctatgttgaagctaccaaacagatcatcaaaactgctcgcttcctcaagtcagaactggaaaatatcaaaggcatctttgtttttgggaatccccaattgtcactcattgctctgggatcccgtgattttgacatctaccgactatcaaacctgatgactgctaaggggtggaacttgaaccagttgcagttcccacccagtattcatttctgcatcacattactacacgcccggaaacgagtagctatacaattcctaaaggacattcgagaatctgtcactcaaatcatgaagaatcctaaagcgaagaccacaggaatgggtgccatctatgccatggcccagacaactgttgacaggaatatggttgcagaattgtcctcagtcttcttggacagcttgtacagcaccgacactgtcacccagggcagccagatgaatggttctccaaaaccccactga

SEQ ID NO: 8 - protein, 568 aa, Homo sapiens
MPSTDLLMLKAFEPYLEILEVYSTKAKNYVNGHCTKYEPWQLIAWSVVWTLLIVWGYEFVFQPESLWSRFKKKCFKLTRKMPIIGRKIQDKLNKTKDDISKNMSFLKVDKEYVKALPSQGLSSSAVLEKLKEYSSMDAFWQEGRASGTVYSGEEKLTELLVKAYGDFAWSNPLHPDIFPGLRKIEAEIVRIACSLFNGGPDSCGCVTSGGTESILMACKACRDLAFEKGIKTPEIVAPQSAHAAFNKAASYFGMKIVRVPLTKMMEVDVRAMRRAISRNTAMLVCSTPQFPHGVIDPVPEVAKLAVKYKIPLHVDACLGGFLIVFMEKAGYPLEHPFDFRVKGVTSISADTHKYGYAPKGSSLVLYSDKKYRNYQFFVDTDWQGGIYASPTIAGSRPGGISAACWAALMHFGENGYVEATKQIIKTARFLKSELENIKGIFVFGNPQLSLIALGSRDFDIYRLSNLMTAKGWNLNQLQFPPSIHFCITLLHARKRVAIQFLKDIRESVTQIMKNPKAKTTGMGAIYAMAQTTVDRNMVAELSSVFLDSLYSTDTVTQGSQMNGSPKPH

SEQ ID NO: 9 - DNA, 1467 bp, Homo sapiens
  CDS (1)...(1467)
atgcctagcacagaccttctgatgttgaaggcctttgagccctacttagagattttggaagtatactccacaaaagccaagaattatgtaaatggacattgcaccaagtatgagccctggcagctaattgcatggagtgtcgtgtggaccctgctgatagtctggggatatgagtttgtcttccagccagagagtttatggtcaaggtttaaaaagaaatgttttaagctcaccaggaagatgcccattattggtcgtaagattcaagacaagttgaacaagaccaaggatgatattagcaagaacatgtcattcctgaaagtggacaaagagtatgtgaaagctttaccctcccagggtctgagctcatctgctgttttggagaaacttaaggagtacagctctatggacgccttctggcaagaggggagagcctctggaacagtgtacagtggggaggagaagctcactgagctccttgtgaaggcttatggagattttgcatggagtaaccccctgcatccagatatcttcccaggactacgcaagatagaggcagaaattgtgaggatagcttgttccctgttcaatgggggaccagattcgtgtggatgtgtgacttctgggggaacagaaagcatactcatggcctgcaaagcatgtcgggatctggcctttgagaaggggatcaaaactccagaaattgtggctccccaaagtgcccatgctgcatttaacaaagcagccagttactttgggatgaagattgtgcgggtcccattgacgaagatgatggaggtggatgtgagggcaatgagaagagctatctccaggaacactgccatgctcgtctgttctaccccacagtttcctcatggtgtaatagatcctgtccctgaagtggccaagctggctgtcaaatacaaaataccccttcatgtcgacgcttgtctgggaggcttcctcatcgtctttatggagaaagcaggatacccactggagcacccatttgatttccgggtgaaaggtgtaaccagcatttcagctgacacccataagctggaaaatatcaaaggcatctttgtttttgggaatccccaattgtcactcattgctctgggatcccgtgattttgacatctaccgactatcaaacctgatgactgctaaggggtggaacttgaaccagttgcagttcccacccagtattcatttctgcatcacattactacacgcccggaaacgagtagctatacaattcctaaaggacattcgagaatctgtcactcaaatcatgaagaatcctaaagcgaagaccacaggaatgggtgccatctatgccatggcccagacaactgttgacaggaatatggttgcagaattgtcctcagtcttcttggacagcttgtacagcaccgacactgtcacccagggcagccagatgaatggttctccaaaaccccactga

SEQ ID NO: 10 - protein, 488 aa, Homo sapiens
MPSTDLLMLKAFEPYLEILEVYSTKAKNYVNGHCTKYEPWQLIAWSVVWTLLIVWGYEFVFQPESLWSRFKKKCFKLTRKMPIIGRKIQDKLNKTKDDISKNMSFLKVDKEYVKALPSQGLSSSAVLEKLKEYSSMDAFWQEGRASGTVYSGEEKLTELLVKAYGDFAWSNPLHPDIFPGLRKIEAEIVRIACSLFNGGPDSCGCVTSGGTESILMACKACRDLAFEKGIKTPEIVAPQSAHAAFNKAASYFGMKIVRVPLTKMMEVDVRAMRRAISRNTAMLVCSTPQFPHGVIDPVPEVAKLAVKYKIPLHVDACLGGFLIVFMEKAGYPLEHPFDFRVKGVTSISADTHKLENIKGIFVFGNPQLSLIALGSRDFDIYRLSNLMTAKGWNLNQLQFPPSIHFCITLLHARKRVAIQFLKDIRESVTQIMKNPKAKTTGMGAIYAMAQTTVDRNMVAELSSVFLDSLYSTDTVTQGSQMNGSPKPH

SEQ ID NO: 11 - protein, 552 aa, C. elegans
MDSVKHTTEIIVDLTKMHYHMINDRLSRYDPVVLVLAAFGGTLVYTKVVHLYRKSEDPILKRMGAYVFSLLRKLPAVRDKIEKELAAEKPKLIESIHKDDKDKQFISTLPIAPLSQDSIMELAKKYEDYNTFNIDGGRVSGAVYTDRHAEHINLLGKIYEKYAFSNPLHPDVFPGARKMEAELIRMVLNLYNGPEDSSGSVTSGGTESIIMACFSYRNRAHSLGIEHPVILACKTAHAAFDKAAHLCGMRLRHVPVDSDNRVDLKEMERLIDSNVCMLVGSAPNFPSGTIDPIPEIAKLGKKYGIPVHVDACLGGFMIPFMNDAGYLIPVFDFRNPGVTSISCDTHKYGCTPKGSSIVMYRSKELHHFQYFSVADWCGGIYATPTIAGSRAGANTAVAWATLLSFGRDEYVRRCAQIVKHTRMLAEKIEKIKWIKPYGKSDVSLVAFSGNGVNIYEVSDKMMKLGWNLNTLQNPAAIHICLTINQANEEVVNAFAVDLEKICEELAAKGEQKADSGMAAMYGMAAQVPKSVVDEVIALYIDATYSAPPSTSN

SEQ ID NO: 12 - DNA, 3162 bp, C. elegans
atggattcggttaagcacacaaccgaaattattgtcgacttgacaaaaatgcactatcacatgataaatgataggtgaattttaaacaaaaattagatatttggaaattactaattcaagattttcagactttctcggtatgatccggttgttctagtgttggccgcttttgggggtacccttgtctatacaaaagtcgtccatttgtaccgaaaaagcgaggatccaattttgaaacggcaagtgttttcttgcgaattttagaaatatcaaaatgaaattttcagcatgggagcttatgtattctcacttcttcgaaaacttccagctgttcgggataaaatcgaaaaagagctggctgctgagaagccaaagcttattgaatcgattcataaggatgataaggacaagcaattcatttccagtttgtttgaacatttattaattaaccaattcattaattctatttttcagctcttcccatcgctccattatctcaggactcaattatggaactggcgaaaaaatatgaggattacaacacatttaacattgacggaggacgagtatctggagcggtttatactgatcgtcatgctgaacacattaatttgcttggaaaggtttagaaattctagaatttttcaaaatcttagctctcaaatatattctcttgtaaatagctacatagtatatcctgtagggaagctttgaatccaattcagatcaggggcgacaaacgattttttccggcaaatcggcaaatcgccggaatggaaatttcctgcaaatcggcaaattgccggaatggaaatttcctgcaagttggcaaattgacggaattgaaatttccggcaaaccgacaaatttccgtaattaaaatttcctgcaaaccggcgaattggcggaattgaaatttcctgcaaaccggcaaattgccgtaattgaaatttcctgcaaaccggcaaattgccggaattgaaatttccggcaaaccggcaaatcggctgaattgaaatttcctgcaaaccggcaaattgcggtaattgaaatttcctgcaaaccggtcagttgccgatttgcctttgcctgaaaaacggcgattgccagaaatattcggcaaattgtggttttgcacatttttctggaaatttcaggcaaaattgtacgcatcctatgaatatccctattaacatcttttttgaaaagtcagtaaattatatgaaaatatctaaagaaaacggggaaaatatttcaaagaggcacagttttatgtgtttccgtcatctaaatagtccctctaaacatttccggcaaatctgatatccggcaaacggcaaatcgggatattgccggaatttaaaatttgccgaacttgtcgacaaaaaaaatgcgccttgaatccgattcagatattcaaaaattgaattttggacgttttagaaatcatttagtttgtcaattttcaagaaatttctagaaaattggatggtttccgccaagaaatattagctacatgaaaataattttgaaactagacatttcttaaaataaaaattgccatcttttatatccagatttacgaaaagtatgcgttctcgaatcccctccaccctgacgtctttccgggagctcgtaaaatggaggcagaacttattcgaatggttctgaacctgtataatggaccagaagattctagtggaagtgtaacttctggtggtactgaaagtattattatggcatgcttttcgtatcggtaagcatttattcaactcttaaaattcaattttgcaaactctacagaaatcgtgcacactctcttggcattgaacatccagttattttggcatgtaaaacagctcacgcggcatttgataaggccgcccatctatgcggaatgcgtcttcgccacgttccagttgattcggataatcgtgtcgatttaaaagaaatggagagactaattgattcgaatgtttgtatgttggttggctcagcgcctaacttcccatcaggcacaattgatccaattccggaaattgctaaggtactggaaattcccgcctcaatatcgcggaaaaaatagagaaatgactgaacaaaattacattgtgagcgggaactctaattgaattcagcaaaaatacgatacttttttctaacttaaaataatttttaaaaaaactcacagatgctagtccaaaaaatggccttttttgattacttaatcgaacgtttacactttcagctcggcaaaaagtatggaatcccggtccacgtggacgcatgtcttggtggattcatgattccatttatgaatgacgccggatacctgattcctgtattcgatttcagaaatcccggtgttacatctatttcgtgtgatactcataaggttggatacagttctatccatttttttccttcaattcaaaatctttcagtacggatgcacaccgaaaggttcatcgattgtcatgtatcgttccaaggaacttcatcacttccagtatttctcggttgccgattggtgtggaggcatctatgccaccccgactattgcaggtttgaagaatgttttagtagcttcaatagaatcaaagagatcccttaggatcccgagctggagccaacactgccgtcgcctgggccacacttttatccttcggtcgagacgaatatgttcgaagatgtgctcaaattgtgaagcatacacgaatgctggccgagaaaattgagaaaatcaaatggatcaagccttatggaaaatcggatgtttcattggtggcgttctccggaaatggtgtgaatatctacgaagtttctgacaaaatgatgaagctcggatggaatttgaacactctgcagaatccagcggcgtatgtttatcaattttatgagttatcagcttgctaaattttttgtttcagaatccacatttgtttgacaatcaatcaagcgaacgaggaagttgtgaatgcgttcgccgtcgaccttgagaagatttgtgaagaactcgctgcaaaaggtgaacaaaaagctgacagtggaatggctgcgatgtatggaatggctgcgcaagtaccaaaatcagtagtggacgaggttatcgctctgtacattgacgcaacttattcagctccaccttcaacttctaattaa

SEQ ID NO: 13 - DNA, 34 bp, Artificial Sequence
  primer
gaggaattcatggattcggttaagcacacaaccg

SEQ ID NO: 14 - DNA, 33 bp, Artificial Sequence
  primer
agcctcgagttaattagaagttgaaggtggagc

SEQ ID NO: 15 - DNA, 1638 bp, Drosophila melanogaster
atgcgtccgttctccggcagcgattgccttaagcccgtcaccgagggcatcaaccgggcgttcggcgccaaggagccctggcaggtggccaccatcacggccaccacggtgctgggaggcgtctggctctggactgtgatctgccaggatgaaaatctttacattcgtggcaagcgtcagttctttaagtttgccaagaagattccagccgtgcgtcgtcaggtggagactgaattggccaaggccaaaaacgacttcgagacggaaatcaaaaagagcaacgcccaccttacctactcggaaactctgcccgagaagggactcagcaaggaggagatcctccgactggtggatgagcacctgaagactggtcactacaactggcgtgatggtcgtgtatctggcgcggtctacggctacaagcctgatctggtggagctcgtcactgaagtgtacggcaaggcctcctacaccaatcccttgcacgcagatcttttcccgggagtttgcaaaatggaggcggaggtagtgcgcatggcatgcaacctgttccatggaaactcagccagctgtggaaccatgaccaccggcggcaccgaatccattgtaatggccatgaaggcgtacagggatttcgctagagagtacaagggaatcaccaggccaaacatcgtggtgcctaagacggtccacgcggccttcgacaagggcggtcagtactttaatatccacgtgcgatccgtggatgtagatccggagacctacgaagtggacattaagaagttcaaacgtgccattaacaggaacacgattctgctggttgggtctgctccgaacttcccctatggaaccatcgatgacatcgaagctatcgccgctttgggcgttaagtacgacattcccgtgcacgtggacgcctgcctgggcagctttgtggtggccttggtccgcaacgccggctataagctgcgtcccttcgactttgaggtcaagggagtgaccagtatctccgctgatacccacaagtatggtttcgcgcccaagggatcatcggtgatcctttactcggacaagaagtacaaggaccatcagttcactgtgactactgactggcctggcggcgtgtatggttctcccacagtcaacggttcccgtgccggaggtattatcgccgcctgctgggctaccatgatgagctttggctatgatggttatctggaagccactaagcgcattgtggatacggcgcgctatatcgagaggggcgttcgcgacatcgatggcatctttatctttggcaagccagctacttcagtgattgccctgggttccaatgtgtttgacattttccggctatcggattcgctgtgcaaactgggctggaacctaaatgcgctgcagtttccatctggtatccacctgtgcgtgacggacatgcacacacagcccggagtcgcggataaattcattgccgatgtgcgcagctgtacggcggagatcatgaaggatcccggccagcccgtcgttggaaagatggctctttacggcatggcacagagcatacccgaccgttcggtgatcggagaagtgactcgcctattcctgcactccatgtactacactcccagccagaaatag

SEQ ID NO: 16 - protein, 545 aa, Drosophila melanogaster
MRPFSGSDCLKPVTEGINRAFGAKEPWQVATITATTVLGGVWLWTVICQDENLYIRGKRQFFKFAKKIPAVRRQVETELAKAKNDFETEIKKSNAHLTYSETLPEKGLSKEEILRLVDEHLKTGHYNWRDGRVSGAVYGYKPDLVELVTEVYGKASYTNPLHADLFPGVCKMEAEVVRMACNLFHGNSASCGTMTTGGTESIVMAMKAYRDFAREYKGITRPNIVVPKTVHAAFDKGGQYFNIHVRSVDVDPETYEVDIKKFKRAINRNTILLVGSAPNFPYGTIDDIEAIAALGVKYDIPVHVDACLGSFVVALVRNAGYKLRPFDFEVKGVTSISADTHKYGFAPKGSSVILYSDKKYKDHQFTVTTDWPGGVYGSPTVNGSRAGGIIAACWATMMSFGYDGYLEATKRIVDTARYIERGVRDIDGIFIFGKPATSVIALGSNVFDIFRLSDSLCKLGWNLNALQFPSGIHLCVTDMHTQPGVADKFIADVRSCTAEIMKDPGQPVVGKMALYGMAQSIPDRSVIGEVTRLFLHSMYYTPSQK

SEQ ID NO: 17 - DNA, 1707 bp, Homo sapiens
  CDS (1)...(1707)
atgcctagcacagaccttctgatgttgaaggcctttgagccctacttagagattttggaagtatactccacaaaagccaagaattatgtaaatggacattgcaccaagtatgagccctggcagctaattgcatggagtgtcgtgtggaccctgctgatagtctggggatatgagtttgtcttccagccagagagtttatggtcaaggtttaaaaagaaatgttttaagctcaccaggaagatgcccattattggtcgtaagattcaagacaagttgaacaagaccaaggatgatattagcaagaacatgtcattcctgaaagtggacaaagagtatgtgaaagctttaccctcccagggtctgagctcatctgctgttttggagaaacttaaggagtacagctctatggacgccttctggcaagaggggagagcctctggaacagtgtacagtggggaggagaagctcactgagctccttgtgaaggcttatggagattttgcatggagtaaccccctgcatccagatatcttcccaggactacgcaagatagaggcagaaattgtgaggatagcttgttccctgttcaatgggggaccagattcgtgtggatgtgtgacttctgggggaacagaaagcatactcatggcctgcaaagcatatcgggatctggcctttgagaaggggatcaaaactccagaaattgtggctccccaaagtgcccatgctgcatttaacaaagcagccagttactttgggatgaagattgtgcgggtcccattgacgaagatgatggaggtggatgtgagggcaatgagaagagctatctccaggaacactgccatgctcgtctgttctaccccacagtttcctcatggtgtaatagatcctgtccctgaagtggccaagctggctgtcaaatacaaaataccccttcatgtcgacgcttgtctgggaggcttcctcatcgtctttatggagaaagcaggatacccactggagcacccatttgatttccgggtgaaaggtgtaaccagcatttcagctgacacccataagtatggctatgccccaaaaggctcatcattggtgttgtatagtgacaagaagtacaggaactatcagttcttcgtcgatacagattggcagggtggcatctatgcttccccaaccatcgcaggctcacggcctggtggcattagcgcagcctgttgggctgccttgatgcacttcggtgagaacggctatgttgaagctaccaaacagatcatcaaaactgctcgcttcctcaagtcagaactggaaaatatcaaaggcatctttgtttttgggaatccccaattgtcagtcattgctctgggatcccgtgattttgacatctaccgactatcaaacctgatgactgctaaggggtggaacttgaaccagttgcagttcccacccagtattcatttctgcatcacattactacacgcccggaaacgagtagctatacaattcctaaaggacattcgagaatctgtcactcaaatcatgaagaatcctaaagcgaagaccacaggaatgggtgccatctatggcatggcccagacaactgttgacaggaatatggttgcagaattgtcctcagtcttcttggacagcttgtacagcaccgacactgtcacccagggcagccagatgaatggttctccaaaaccccactga

SEQ ID NO: 18 - protein, 568 aa, Homo sapiens
MPSTDLLMLKAFEPYLEILEVYSTKAKNYVNGHCTKYEPWQLIAWSVVWTLLIVWGYEFVFQPESLWSRFKKKCFKLTRKMPIIGRKIQDKLNKTKDDISKNMSFLKVDKEYVKALPSQGLSSSAVLEKLKEYSSMDAFWQEGRASGTVYSGEEKLTELLVKAYGDFAWSNPLHPDIFPGLRKIEAEIVRIACSLFNGGPDSCGCVTSGGTESILMACKAYRDLAFEKGIKTPEIVAPQSAHAAFNKAASYFGMKIVRVPLTKMMEVDVRAMRRAISRNTAMLVCSTPQFPHGVIDPVPEVAKLAVKYKIPLHVDACLGGFLIVFMEKAGYPLEHPFDFRVKGVTSISADTHKYGYAPKGSSLVLYSDKKYRNYQFFVDTDWQGGIYASPTIAGSRPGGISAACWAALMHFGENGYVEATKQIIKTARFLKSELENIKGIFVFGNPQLSVIALGSRDFDIYRLSNLMTAKGWNLNQLQFPPSIHFCITLLHARKRVAIQFLKDIRESVTQIMKNPKAKTTGMGAIYGMAQTTVDRNMVAELSSVFLDSLYSTDTVTQGSQMNGSPKPH

SEQ ID NO: 19 - protein, 490 aa, Drosophila melanogaster
FRSSNDYGVNLQTAEMWHHTIRKHKRGNGSSSPADCGKQLLILLNPKSGSGKGRELFQKQVAPLLTEAEVQYDLQITTHPQYAKEFVRTRRDLLTRYSGIVVASGDGLFYEVLNGLMERMDWRRACRELPLGIIPCGSGNGLAKSVAHHCNEPYEPKPILHATLTCMAGKSTPMDVVRVELATRDKHFVMYSFLSVGWGLIADIDIESERLRSIGAQRFTLWAIKRLIGLRSYKGRVSYLLGKGKKEPPVEAARELPAESTAAGIRSSLPLNAGEFHDLPEEEEGEAVLDGEQFADAISLDRSVYRQHADSWHSAMSRRTAYYSLGGPSMRSNRSRMSISQRIEAANAEFAERVPTGTIPPLQMPLLSSDGWICEDGDFVMVHAAYTTHLSSDVFFAPESRLDDGLIYLVIIRRGVSRHQLLNFMLNLNAGTHLPIGEDPFIKVVPCRAFRIEPSSSDGILVVDGERVEYGPIQAEVMPGLINVMTTSGQ

SEQ ID NO: 20 - protein, 524 aa, Drosophila melanogaster
FRSFDTFEDNMREADRWYRSLRWQLHRTLEEIFVAPTVDERRRRVLVLLNPKSGSGDAREVFNMHVTPVLNEAEVPYDLYVTKHSNFAIEFLSTRCLDAWCCVVAVGGDGLFHEIVNGLLQRQDWAHVLPHLALGIIPCGSGNGLARSIAHCYNKPVLGAALTVISGRSSPMDVVRVQLQSRSLYSFLSIGWGLISDVDIESERIRMLGYQRFTVWTLYRLVNLRTYNGRISYLLTDHEVSSTHSATGYAAQRRMQSSRSCNTHIDMLNGPAPIYHSSAEYLPQEFADVISLETSINQSFRSRCDSWLSGGSRRSFYYSISESIYHSLADESEFAGLAAASLENRQQNYGPASELPDLNEPLSEDQGWLVEEGEFVMMHAVYQTHLGIDCHFAPKAQLNDGTIYLILIRAGISRPHLLSFLYNMSSGTHLPESHDDHVKVLPVRAFRLEPYDNHGIITVDGERVEFGPLQAEVLPGIARVMVPNVSTFRFQSATLQHGIPVCIPVRKRFVLYNMSSEELAPINE

SEQ ID NO: 21 - protein, 368 aa, Homo sapiens
VLVLLNPRGGKGKALQLFRSHVQPLLAEAEISFTLMLTERRNHARELVRSEELGRWDALVVMSGDGLMHEVVNGLMERPDWETAIQKPLCSLPAGSGNALAASLNHYAGYEQVTNEDLLTNCTLLLCRRLLSPMNLLSLHTASGLRLFSVLSLAWGFIADVDLESEKYRRLGEMRFTLGTFLRLAALRTYRGRLAYLPVGRVGSKTPASPVVVQQGPVDAHLVPLEEPVPSHWTVVPDEDFVLVLALLHSHLGSEMFAAPMGRCAAGVMHLFYVRAGVSRAMLLRLFLAMEKGRHMEYECPYLVYVPVVAFRLEPKDGKGVFAVDGELMVSEAVQGQVHPNYFWMVSGCVEPPPSWKPQQMPPPEEPL

SEQ ID NO: 22 - DNA, 21 bp, Artificial Sequence
  Oligonucleotide used to achieve knockdown of mouse SPL using the pSilencer adeno 1.0-CMV system
tatgagccctggcagctcatt

SEQ ID NO: 23 - DNA, 50 bp, Artificial Sequence
  Top oligomer used for knockdown of human SPL in HUVECs, designed using Dharmacon siDesign Center
gcatactgatggcctgcaattcaagagattgcaggccatcagtatgctta

SEQ ID NO: 24 - DNA, 55 bp, Artificial Sequence
  Bottom oligomer used for knockdown of human SPL in HUVECs, designed using Dharmacon siDesign Center
ctagtaagcatactgatggcctgcaatctcttgaattgcaggccatcagtatgcc

SEQ ID NO: 25 - DNA, 24 bp, Artificial Sequence
  primer for SPL genotyping
cgctcagaaggctctgagtcatgg

SEQ ID NO: 26 - DNA, 24 bp, Artificial Sequence
  primer for SPL genotyping
ccaagtgtacctgctaagttccag

SEQ ID NO: 27 - DNA, 24 bp, Artificial Sequence
  primer for SPL genotyping
catcaaggaaaccctggactactg

(19) United States
(12) Patent Application Publication
(10) Pub. No.: US 2008/0248032 A1
(43) Pub. Date: Oct. 9, 2008

(54) COMPOSITIONS AND METHODS FOR PROTECTION AGAINST CARDIAC AND/OR CENTRAL NERVOUS SYSTEM TISSUE INJURY BY INHIBITING SPHINGOSINE-1-PHOSPHATE LYASE

(75) Inventor: Julie D. Saba, Oakland, CA (US)

Correspondence Address: SEED INTELLECTUAL PROPERTY LAW GROUP PLLC, 701 FIFTH AVE, SUITE 5400, SEATTLE, WA, 98104, US

(73) Assignee: CHILDREN'S HOSPITAL & RESEARCH CENTER AT OAKLAND, Oakland, CA (US)

(21) Appl. No.: 11/943,430

(22) Filed: Nov. 20, 2007

Related U.S. Application Data
(60) Provisional application No. 60/860,394, filed on Nov. 21, 2006.

(51) Int. Cl. A61K 31/4164; A61K 39/395; A61P 9/00; A61K 31/7088

(52) U.S. Cl. 424/133.1; 514/400; 424/139.1; 514/44

(57) ABSTRACT

The present invention relates generally to the prevention and/or treatment of cardiac and stroke injury. In particular, the present invention provides compositions and methods for preventing and treating tissue injury in cardiac and stroke settings and injury due to ischemia/reperfusion, hypoxia, cardiotoxicity of certain therapeutic regimens, and other causes, by administering an agent that inhibits sphingosine-1-phosphate lyase (SPL) activity.

### CROSS-REFERENCE TO RELATED APPLICATION

This application claims the benefit under 35 U.S.C. § 119(e) of U.S. Provisional Patent Application No. 60/860,394 filed Nov. 21, 2006, where this provisional application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 200116.408_SEQUENCE_LISTING.txt. The text file is 96 KB, was created on Nov. 20, 2007, and is being submitted electronically via EFS-Web, concurrent with the filing of the specification.

### BACKGROUND OF THE INVENTION

1. Field of the Invention

The present invention relates generally to the prevention and/or treatment of cardiac and stroke injury caused by a variety of factors and/or conditions. In particular, the present invention provides compositions and methods for preventing and treating injury due to ischemia/reperfusion injury, hypoxia, cardiotoxicity of certain therapeutic regimens, and other causes.

2. Description of the Related Art

Limitation of blood flow to the heart causes ischemia (cell starvation secondary to a lack of oxygen) of the myocardial cells. Myocardial infarction (commonly called a heart attack) is caused when myocardial cells die from lack of oxygen. It leads to heart muscle damage, heart muscle death and later scarring without heart muscle regrowth. Despite much research and current medicines, heart attacks are a leading cause of death in men and women in the US. As such, there remains a need in the art for effective agents that inhibit and treat cardiac injury such as injury due to ischemia/reperfusion resulting from coronary heart disease. The present invention provides this and other related advantages.

Cardiovascular side effects are also associated with radiation therapy, chemotherapy and immune-based therapy and limit the dosages of such therapies that can be safely delivered to patients. These side effects comprise a significant proportion of the serious morbidity and mortality associated with cancer therapy (Yeh et al., Circulation 2004; 109: 3122-3131). There is abundant evidence that doxorubicin, one of the most widely used antitumor agents, causes cardiomyopathy in a dose-dependent manner, leading to heart failure in cancer patients treated with this agent (Yi et al., Am J Physiol Heart Circ Physiol 2006; 290: H1098-1102). Echocardiography has demonstrated that up to 90% of children with anthracycline cardiomyopathy manifest some changes during the first year of therapy (Krischer et al., J Clin Oncol 1997; 15: 1544-1552). Acute cardiotoxicites are associated with anthracyclines (Berry et al., Pediatr Blood Cancer 2005; 44: 630-637), radiation, and trastuzumab (Slamon et al., N Engl J Med 2001; 344: 783-792). In addition, an increase in the risk of late cardiovascular effects, including coronary heart disease, myocardial infarction, and angina pectoris, is now appreciated in cancer patients receiving combination therapies that include cisplatin, cytoxan, etoposide, vinblastine and/or bleomycin and mediastinal or subdiaphragmatic radiation (van den Belt-Dusebout et al., J Clin Oncol 2006; 24: 467-475). These late effects are becoming increasingly evident as patients with breast cancer, Hodgkins's lymphoma and pediatric malignancies are living longer after curative treatment (Steingart, J Clin Oncol 2005; 23: 9051-9052; Greving et al., J Pediatr Oncol Nurs 2005; 22: 38-47). Therefore, there is a need in the art for interventions that protect against cardiovascular disease and cardiovascular toxicities of existing therapeutic modalities for the overall management of cancer patients.

Stroke occurs when the blood supply to part of the brain is suddenly interrupted or when a blood vessel in the brain bursts, spilling blood into the spaces surrounding brain cells. Brain cells die when they no longer receive oxygen and nutrients from the blood or there is sudden bleeding into or around the brain. The symptoms of a stroke include sudden numbness or weakness, especially on one side of the body; sudden confusion or impaired speech or speech comprehension; sudden trouble seeing in one or both eyes; sudden difficulty with walking, dizziness, or loss of balance or coordination; or sudden severe headache with no known cause. There are two forms of stroke: ischemic—i.e., blockage of a blood vessel supplying the brain, and hemorrhagic—i.e., bleeding into or around the brain.

Therapies to prevent a first or recurrent stroke are based on treating an individual's underlying risk factors for stroke, such as hypertension, atrial fibrillation, and diabetes. Acute stroke therapies try to stop a stroke while it is happening by quickly dissolving the blood clot causing an ischemic stroke or by stopping the bleeding of a hemorrhagic stroke. The most popular classes of drugs used to prevent or treat stroke are antithrombotic (antiplatelet agents and anticoagulants) and thrombolytics. However, there remains a need in the art for effective agents that inhibit and treat stroke injury such as injury due to ischemia/reperfusion. The present invention provides this and other related advantages.

### BRIEF SUMMARY OF THE INVENTION

One aspect of the present invention provides a method for reducing or preventing cardiac injury in a subject known to have, or to be at risk for sustaining, cardiac injury, comprising administering to the subject an agent that inhibits sphingosine-1-phosphate lyase (SPL) activity and thereby reducing or preventing cardiac injury in the subject.

Another aspect of the invention provides a method for reducing or preventing stroke injury in a subject known to have, or to be at risk for sustaining, stroke injury, comprising administering to the subject an agent that inhibits sphingosine-1-phosphate lyase (SPL) activity and thereby reducing or preventing stroke injury in the subject.

A further aspect provides a method for preventing or reducing tissue injury due to organ transplantation in a subject, comprising administering to the subject an agent that inhibits sphingosine-1-phosphate lyase (SPL) activity and thereby preventing or reducing tissue injury due to organ transplantation.

In certain embodiments of the methods provided herein the agent that inhibits SPL activity comprises 2-acetyl-4-tetrahydroxybutylimidazole (THI) or a metabolite thereof. The structure of THI is shown below.

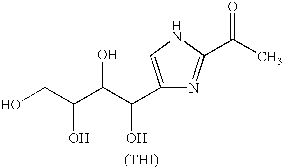

In this regard, metabolites include but are not limited to glucuronidated, phosphorylated, and methylated THI products (see e.g. V. Sarvesh et al. 2001 Drug Metabolism and Disposition 29:1290; Huskey et al., 1994 Drug Metabolism and Disposition 22: 651). In certain embodiments, glucuronidation occurs either on the imidazole ring or one of the four hydroxy groups. In a further embodiment, a THI metabolite may comprise a carbonyl compound (e.g., a ketone or an aldehyde) resulting from oxidizing any of the hydroxy groups (e.g., via P450 catalyzed oxidation). In another embodiment, a metabolite of THI comprises an alcohol-containing compound resulting from the carbonyl reduction (e.g., by carbonyl reductases) of the ketone.

In other embodiments, the methods further comprise administering to the subject at least one of a beta-blocker and an antioxidant. In this regard, the beta-blocker may be any one or combination of acebutolol, betaxolol, carteolol, labetalol, metoprolol or propranolol. The antioxidant may be ascorbic acid or sodium bisulfite, or a combination thereof. It should be noted that other antioxidants known to the skilled artisan are also contemplated for use herein.

In another embodiment, the agents useful in the methods herein comprise at least one antibody that specifically binds to a human SPL polypeptide. In this regard, human SPL polypeptides are known to the skilled artisan and are available in public databases such as GENBANK. Illustrative human SPL polypeptides include the amino acid sequences as set forth in any one of SEQ ID NOS:8, 10 and 18. In certain embodiments, the antibodies useful herein may be polyclonal antibodies, monoclonal antibodies, chimeric antibodies, humanized antibodies, Fab fragments, Fab′ fragments, (Fab′)₂ fragments, Fd fragments, Fv fragments, scFv, dAb or diabodies.

In certain embodiments, the agent that inhibits SPL activity comprises an antisense nucleic acid that specifically hybridizes to a human SPL encoding polynucleotide. Polynucleotides encoding human SPL polypeptides are known to the skilled artisan. Illustrative polynucleotides are set forth in SEQ ID NOS:7, 19 and 17.

In a further embodiment of an agent that inhibits SPL activity comprises an RNAi molecule that interferes with expression of a SPL polypeptide having SPL activity, wherein the SPL polypeptide having SPL activity may be (i) a polypeptide that comprises the amino acid sequence as set forth in any one of SEQ ID NOS:8, 10 and 18, (ii) a polypeptide that is encoded by the nucleotide sequence set forth in any one of SEQ ID NOS:7, 19 and 17, or (iii) a polypeptide that is encoded by a polynucleotide that is capable of hybridizing under moderately stringent conditions to a nucleic acid having the nucleotide sequence set forth in any one of SEQ ID NOS:7, 19 and 17, or a complementary sequence thereto.

In certain embodiments the agent that inhibits SPL activity comprises a ribozyme that interferes with expression of a SPL polypeptide having SPL activity, wherein the SPL polypeptide having SPL activity may be (i) a polypeptide that comprises the amino acid sequence as set forth in any one of SEQ ID NOS:8, and 18, (ii) a polypeptide that is encoded by the nucleotide sequence set forth in any one of SEQ ID NOS:7, 19 and 17, or (iii) a polypeptide that is encoded by a polynucleotide that is capable of hybridizing under moderately stringent conditions to a nucleic acid having the nucleotide sequence set forth in any one of SEQ ID NOS:7, 19 and 17, or a complementary sequence thereto.

In one embodiment, the cardiac injury comprises acute ischemia/reperfusion injury. In this regard, the acute ischemia/reperfusion injury may be due to one or more events such as coronary obstruction, cardiac percutaneous intervention, coronary artery bypass surgery, cardiopulmonary bypass or non-cardiac surgery. In another embodiment, the cardiac injury comprises an injury resulting from surgical repair of congenital heart disease, an injury resulting from closure of septal defects by percutaneous means, an injury resulting from percutaneous mitral valve repair or mitral valvulotomy, an injury resulting from hypoxia, an injury resulting from hypoxia with reperfusion, an injury resulting, from hypoxia with reoxygenation, an injury resulting from myocardial infarction, an injury resulting from acute congestive heart failure, chronic congestive heart failure, myocarditis or from cardiotoxicity of a drug. In this regard, the drug may be anti-Her2 antibodies or anthracyclines. In one embodiment the cardiac injury comprises injury resulting from cardiotoxicity of radiation treatment. In a further embodiment, the cardiac injury comprises injury resulting from heart transplantation or injury resulting from iron overload.

In another embodiment, the stroke injury comprises acute ischemia/reperfusion injury, injury resulting from hypoxia, injury resulting from hypoxia with reperfusion, injury resulting from hypoxia with reoxygenation, injury accompanying toxic dementia, injury resulting from vascular dementia, injury accompanying Alzheimer's disease, or injury due to neurotoxicity, or injuries caused by a combination of any of these.

A further aspect of the invention provides a method for reducing or preventing ischemia/reperfusion injury in a tissue in a mammal comprising, administering to said mammal an agent that inhibits SPL activity.

These and other aspects of the present invention will become apparent upon reference to the following detailed description. To this end, various references are set forth herein which describe in more detail certain background information, procedures, compounds and/or compositions, and are each hereby incorporated by reference as if set forth in their entirety.

### BRIEF DESCRIPTION OF THE SEQUENCE IDENTIFIERS

SEQ ID NO:1 is the determined cDNA sequence of S. cerevisiae SPL.

SEQ ID NO:2 is the amino acid sequence of S. cerevisiae SPL encoded by the polynucleotide sequence set forth in SEQ ID NO:1.

SEQ ID NO:3 is the determined cDNA sequence of C. elegans SPL.

SEQ ID NO:4 is the amino acid sequence of C. elegans SPL encoded by the polynucleotide sequence set forth in SEQ ID NO:3.

SEQ ID NO:5 is the determined cDNA sequence of the mouse SPL.

SEQ ID NO:6 is the amino acid sequence of mouse SPL encoded by the polynucleotide sequence set forth in SEQ ID NO:5.

SEQ ID NO:7 is the determined cDNA sequence of the full-length human SPL.

SEQ ID NO:8 is the amino acid sequence of human SPL encoded by the polynucleotide sequence set forth in SEQ ID NO:7.

SEQ ID NO:9 is the determined cDNA sequence of a human SPL with a deletion.

SEQ ID NO:10 is the amino acid sequence of a human SPL with a deletion, encoded by the polynucleotide sequence set forth in SEQ ID NO:9.

SEQ ID NO:11 is the amino acid sequence of C. elegans SPL encoded by the polynucleotide sequence set forth in SEQ ID NO:12.

SEQ ID NO:12 is the determined cDNA sequence of a C. elegans SPL.

SEQ ID NO:13 is a PCR primer.

SEQ ID NO:14 is a PCR primer.

SEQ ID NO:15 is the determined cDNA sequence encoding the Drosophila melanogaster SPL.

SEQ ID NO:16 is the amino acid sequence of the Drosophila melanogaster SPL, encoded by the cDNA sequence set forth in SEQ ID NO:15.

SEQ ID NO:17 is the determined cDNA sequence of a human SPL as set forth in Genbank Accession No: AF144638.

SEQ ID NO:18 is the amino acid sequence of a human SPL encoded by the polynucleotide sequence provided in SEQ ID NO:17.

SEQ ID NO:19 is the amino acid sequence of a first Drosophila melanogaster SK protein.

SEQ ID NO:20 is the amino acid sequence of a second Drosophila melanogaster SK protein.

SEQ ID NO:21 is the amino acid sequence of a human SK protein.

SEQ ID NO:22 is an oligonucleotide used to achieve knockdown of mouse SPL using the pSilencer adeno 1.0-CMV system (see Example 3).

SEQ ID NO:23 is a top oligomer used for knockdown of human SPL in HUVECs, designed using Dharmacon siDesign Center (see Example 3).

SEQ ID NO:24 is a bottom oligomer used for knockdown of human SPL in HUVECs, designed using Dharmacon siDesign Center (see Example 3).

SEQ ID NO:25 is a primer for SPL genotyping as described in Example 4.

SEQ ID NO:26 is a primer for SPL genotyping as described in Example 4.

SEQ ID NO:27 is a primer for SPL genotyping as described in Example 4.

### BRIEF DESCRIPTION OF THE SEVERAL VIEWS OF THE DRAWINGS

FIG. 1, is a bar graph showing cell survival in the presence of doxorubicin in cells overexpressing SPL.

FIG. 2. is a bar graph showing survival of HEK293 kidney cell stably overexpressing human SPL (hSPL) in the presence of the cardiotoxic agent daunorubicin.

FIG. 3A shows a schematic representation of Langendorff hanging heart protocols used in experiments as described in Example 12.

FIG. 3B is a bar graph showing SPL activity in ischemic tissues as compared to baseline SPL activity levels.

### DETAILED DESCRIPTION OF THE INVENTION

The present invention is generally directed to the reduction and/or prevention of cardiac and stroke injury and other tissue injury caused by a variety of conditions or factors as described herein. In particular, certain invention embodiments described herein provide compositions and methods for reducing (e.g., decreasing in a statistically significant manner) or preventing certain types of tissue injury that would otherwise result from clinical or physiological insults such as ischemia/reperfusion events, hypoxia, cardiotoxicity of certain therapeutic regimens, and other causes.

These and related embodiments are based in part on the unexpected discovery that inhibition of the cytoplasmic metabolic enzyme sphingosine-1-phospate lyase (SPL) can prevent or reduce injuries to cardiac tissue and/or to central nervous system (CNS) tissue that might otherwise occur in the course of and/or as sequelae to stroke, ischemia/reperfusion, cardiotoxic therapies, neurotoxicity, excitotoxicity, organ transplantation, trauma including trauma related to surgery, coronary heart disease, atherosclerosis, myocardial infarction, hypoxia, congestive heart failure, iron overload and/or other causes. The present invention provides compositions and methods for treating any cardiac failure resulting from cellular injury, from any apoptotic or necrotic process, including congestive heart failure from any number of infectious, toxic, genetic/inborn errors or other metabolic causes. Accordingly and as disclosed herein, there are provided compositions and methods for beneficially treating cardiac injury and stroke, and other related conditions, through intervention by inhibiting SPL.

Agents that Inhibit SPL

As also noted above, certain presently disclosed embodiments provide compositions and methods that include the use of an agent that inhibits the expression (transcription or translation), stability and/or activity of an SPL polypeptide.

Agents that alter SPL activity are described herein and additional suitable agents for use according to the present embodiments may be identified according to routine methodologies, such as those described herein and in the incorporated references. For instance, methods of detecting SPL activity, and of screening compound libraries for agents that alter SPL activity, including polynucleotide sequences for the production of nucleic acid molecules that encode SPL polypeptides and the production of SPL polypeptides therefrom, are disclosed in U.S. application Ser. No. 10/348,052, U.S. application Ser. No. 10/622,011, and PCT/US2003/01739, including in publications cited therein (e.g., Van Veldhoven et al., J Biol Chem 1991; 266: 12502-07) and elsewhere. For embodiments that relate to molecular biology methodologies, compositions and methods well known to those of ordinary skill in the art are described for example, in Sambrook et al., Molecular Cloning: A Laboratory Manual, Cold Spring Harbor Laboratories, Cold Spring Harbor, N.Y., 1989; Ausubel et al., Current Protocols in Molecular Biology, Greene Publ. Assoc. Inc. & John Wiley & Sons, Inc., Boston, Mass., 1993); Maniatis et al. (Molecular Cloning, Cold Spring Harbor Laboratory, Plainview, N.Y., 1982) and elsewhere.

Certain embodiments as provided herein expressly contemplate methods for preventing or treating cardiac, stroke, and ischemia/reperfusion injury in a subject (such as to the kidney and other organs) that comprises administering an agent that alters (i.e., increases or decreases in a statistically significant manner, and in certain preferred embodiments decreases) SPL activity, wherein the agent is 2-acetyl-4-tetrahydroxybutylimidazole (THI), or a metabolite thereof. The structure of THI is shown below.

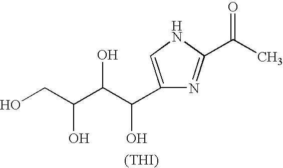

Metabolites of THI include but are not limited to glucuronidated, phosphorylated, and methylated THI products (see e.g. V. Sarvesh et al., 2001 Drug Metabolism and Disposition 29:1290; Huskey et al, 1994 Drug Metabolism and Disposition 22: 651). In certain embodiments, glucuronidation occurs either on the imidazole ring or one of the four hydroxy groups. In a further embodiment, a THI metabolite may comprise a carbonyl compound (e.g., a ketone or an aldehyde) resulting from oxidizing any of the hydroxy groups (e.g., via P450 catalyzed oxidation). In another embodiment, a metabolite of THI comprises an alcohol-containing compound resulting from the carbonyl reduction (e.g., by carbonyl reductases) of the ketone.

THI is a component of caramel food colorant III used in food products and has been shown to suppress immunity by increasing S1P in tissues of the immune system (Hla, Science 2005; 309:1682-1683; Schwab et al. Science 2005; 309:1735; see also Lawrence J F, et al., J Chromatogr 1989; 466:421-6; Morita Y, et al., Nat Med 2000; 6(10):1109-14; Yoshimura Y, et al., Cardiovasc Drugs Ther 2004; 18(6):433-40; Vessey D, et al., Med Sci Monit 2006; 12:BR318-24). Certain other embodiments are not so limited and may include embodiments wherein methods for preventing or treating cardiac, stroke, and ischemia/reperfusion injury comprise administering THI, or a metabolite thereof, optionally in combination with one or more additional agents, such as an agent that alters SPL activity and/or an agent that alters SK activity and/or an agent that alters a level of S1P or an interaction between S1P and an S1P receptor such as S1P₁ (or another G protein-coupled S1P receptor), or another agent.

In certain other embodiments methods for preventing or treating cardiac, stroke, and ischemia/reperfusion injury in a subject is expressly contemplated that comprises administering an agent that alters SPL activity wherein the agent is at least one of deoxypyridoxine, C₈-cyclopropenylceramide and C₁₆-cyclopropenylceramide, each of which has been shown to be capable of altering SPL activity (see, e.g., Triola et al., Mol Pharmacol 2004; 66: 1671; see also Matreya LLC 2005 Catalog (Pleasant Gap, Pa.), Cat. Nos. 1886 and 1887). FTY720 is a further agent that inhibits SPL activity and is contemplated for use in the methods described herein (see e.g., P. Bandhuvula et al., 2005 J. Biol. Chem. 280 33697-33700). Other SPL inhibitors contemplated for use in the methods described herein include semicarbazide (see e.g., P. Bandhuvula et al., 2007 J. Lipid Research 48, A Rapid Fluorescence Assay for Sphingosine-1-phosphate Lyase Enzyme Activity) and GT11 (see e.g., Triola G. et al., 2004 Mol Pharmacol 66:1671-1680).

Without being bound by theory, it is thought that THI may inhibit SPL activity by interfering with the ability of the SPL cofactor B6 (pyridoxal 5′-phosphate) to bind to SPL and allow the enzyme to function properly. As such, inhibitors of the B6 (pyridoxal 5′-phosphate)-dependent enzymes are also contemplated for use as SPL inhibitors for the methods described herein (see e.g., Amadasi A., et al. 2007 Current Med. Chem. 14(12):1291-324).

As also provided herein, certain contemplated embodiments relate to a method for preventing or treating cardiac, stroke, or ischemia/reperfusion injury in a subject by administering an agent that decreases SPL activity, which in certain embodiments may involve an agent that decreases SPL activity by directly binding to SPL, while in certain other embodiments an agent that decreases SPL activity may do so indirectly, for example, by interacting with other cellular molecular components that exert an effect on SPL activity. Certain contemplated embodiments relate to an agent that is capable of decreasing SPL activity by causing a decreased expression level of SPL.

Compositions and methods directed to altering (e.g., increasing or decreasing with statistical significance) SPL expression levels are described in U.S. Pat. No. 6,423,527; U.S. Pat. No. 6,569,666; U.S. Pat. No. 6,495,359; U.S. application Ser. No. 10/053,510; U.S. application Ser. No. 10/286,175; U.S. application Ser. No. 10/197,073; U.S. application Ser. No. 10/348,052; U.S. application Ser. No. 10/622,011; and PCT/US2003/01739, wherein can be found abundant disclosure describing nucleic acid molecules that encode SPL polypeptides, including SPL-encoding polynucleotides (and nucleotides that hybridize thereto under moderately stringent conditions, which may be, e.g., prewashing in a solution of 5×SSC, 0.5% SDS, 1.0 mM EDTA (pH 8.0); hybridizing at 50-65° C., 5×SSC, overnight; followed by washing twice at 65° C. for 20 minutes with each of 2×, 0.5× and 0.2×SSC containing 0.1% SDS) and encoded SPL polypeptide sequences, and wherein also can be found description of antibodies that specifically bind to a translated polypeptide product of an SPL-encoding nucleic acid molecule. Certain embodiments contemplated herein thus include the use of all or a portion of such an SPL-encoding polynucleotide and/or a polynucleotide that hybridizes thereto under moderately stringent conditions, for example, a polynucleotide that comprises all or a portion of the nucleotide sequence set forth in any one of SEQ ID NOs:1, 3, 5, 7, 9, 12, 15, and 17.

### SPL/S1P

The present invention thus relates generally to compositions and methods for inhibiting sphingosine-1-phosphate lyase (SPL) activity. Inhibiting SPL activity is beneficial in a number of disease settings including preventing or reducing cardiac, stroke, and ischemia/reperfusion injury.

Sphingosine-1-phosphate (S1P) is an endogenous lipid metabolite that acts through established signaling pathways to enhance cell survival after radiation, chemotherapy and other stressful stimuli. S1P is irreversibly degraded by the intracellular enzyme S1P lyase (SPL) (Van Veldhoven P P, Methods Enzymol 2000; 311: 244-254). Overexpression of SPL in human cells lowers intracellular S1P, increases ceramide and potentiates apoptosis in response to stress and cytotoxic therapy through a mechanism involving p53 and p38 MAP kinase (Oskouian B, et al., Proc Natl Acad Sci USA. 2006 Nov. 14; 103(46):17384-17389; Reiss U, et al., J Biol Chem 2004; 279: 1281-1290). Further, SPL expression is downregulated in cancer (see U.S. patent application Ser. No. 10/622,011). Thus, increasing SPL activity in cancer cells may be a useful strategy for enhancing tumor responses to therapeutic intervention. Conversely, inhibiting SPL leads to accumulation of S1P, thereby attenuating apoptotic responses.

Agents that decrease the expression or activity of endogenous SPL polypeptides are encompassed within certain presently disclosed embodiments. Such agents may be identified using methods described herein and used, for example, in therapy for the prevention and treatment of cardiac injury, stroke injury, tissue injury due to organ transplantation, and generally any ischemia/reperfusion injury in a tissue.

As used herein, the term “polypeptide” encompasses amino acid chains of any length, including full length endogenous (i.e., native) SPL proteins and variants of endogenous sequences. Illustrative SPL proteins are provided in SEQ ID NOs:2, 4, 6, 8, 10, 11, 16, and 18. “Variants” are polypeptides that differ in sequence from a native SPL only in substitutions, deletions and/or other modifications, such that the variant retains SPL activity, which may be determined using a representative method described herein SPL polypeptide variants generally encompassed by the present invention will typically exhibit at least about 70%, 75%, 80%, 85%, 90%, 91%, 92%, 93%, 94%, 95%, 96%, 97%, 98%, or 99% or more identity along its length, to an SPL polypeptide sequence set forth herein. Within an SPL polypeptide variant, amino acid substitutions are preferably made at no more than 50% of the amino acid residues in the native polypeptide, and more preferably at no more than 25% of the amino acid residues. Such substitutions are preferably conservative. A conservative substitution is one in which an amino acid is substituted for another amino acid that has similar properties, such that one skilled in the art of peptide chemistry would expect the secondary structure and hydropathic nature of the polypeptide to be substantially unchanged. In general, the following amino acids represent conservative changes: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. Substitutions, deletions and/or amino acid additions may be made at any location(s) in the polypeptide, provided that the modification does not diminish the SPL activity of the variant. Thus, a variant may comprise only a portion of a native SPL sequence. In addition, or alternatively, variants may contain additional amino acid sequences (such as, for example, linkers, tags and/or ligands), preferably at the amino and/or carboxy termini. Such sequences may be used, for example, to facilitate purification, detection or cellular uptake of the polypeptide.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the MEGALIGN™ program in the Lasergene™ suite of bioinformatics software (DNASTAR, Inc., Madison, Wis.), using default parameters. This program embodies several alignment schemes described in the following references: Dayhoff, M. O., A model of evolutionary change in proteins—Matrices for detecting distant relationships, in Dayhoff, M. O. (ed.) Atlas of Protein Sequence and Structure, National Biomedical Research Foundation, Washington D.C. 1978; 5(3): 345-358; Hein J, Unified Approach to Alignment and Phylogenes, Methods in Enzymology 1990; 183: 626-645, Academic Press, Inc., San Diego, Calif.; Higgins D G, et al., CABIOS 1989; 5:151-153; Myers E W, et al., CABIOS 1988; 4:11-17; Robinson, E D, Comb Theor 1971; 11: 105; Saitou N, et al., Mol Biol Evol 1987; 4: 406-425; Sneath P H A, et al., Numerical Taxonomy—the Principles and Practice of Numerical Taxonomy, 1973; Freeman Press, San Francisco, Calif.; Wilbur W J et al., Proc Natl Acad, Sci. USA 1983; 80: 726-730.

Alternatively, optimal alignment of sequences for comparison may be conducted by the local identity algorithm of Smith and Waterman. Add. APL. Math 1981; 2: 482, by the identity alignment algorithm of Needleman and Wunsch, J Mol Biol 1970; 48: 443, by the search for similarity methods of Pearson and Lipman, Proc Natl Acad Sci USA 1988; 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

Preferred examples of algorithms that are suitable for determining percent sequence identity and sequence similarity are the BLAST and BLAST 2.0 algorithms, which are described in Altschul et al., Nucl Acids Res 1977; 25: 3389-3402 and Altschul et al., J Mol Biol 1990; 215: 403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

SPL polypeptide sequence variants may also be identified based on sequence differences from the sequences disclosed herein that do not compromise the three-dimensional structure of the enzyme. For example, determination of the three-dimensional structures of representative SPL polypeptide variants may be made through routine methodologies such that substitution of one or more amino acids with selected natural or non-natural amino acids can be virtually modeled for purposes of determining whether a so derived structural variant retains the space-filling properties of disclosed species. See, for example, Bradley et al., Science 309: 1868-1871 (2005); Schueler-Furman et al., Science 310:638 (2005); Qian et al., Nature 450:259 (2007); Dietz et al. Proc. Nat. Acad. Sci. USA. 103:1244 (2006). Some additional non-limiting examples of computer algorithms that may be used for these and related embodiments, such as for identification of SPL polypeptide variants as provided herein, include Desktop Molecular Modeler (See, for example, Agboh et al., J. Biol. Chem., 279, 40: 41650-57 (2004)), which allows for determining atomic dimensions from spacefilling models (van der Waals radii) of energy-minimized conformations; GRID, which seeks to determine regions of high affinity for different chemical groups, thereby enhancing binding, Monte Carlo searches, which calculate mathematical alignment, and CHARMM (Brooks et al. (1983) J. Comput. Chem. 4:187-217) and AMBER (Weiner et al (1981) J. Comput. Chem. 106: 765), which assess force field calculations, and analysis (see also, Eisenfield et al. (1991) Am. J. Physiol. 261:C376-386; Lybrand (1991) J. Pharm. Belg. 46:49-54; Froimowitz (1990) Biotechniques 8:640-644; Burbam et al. (1990) Proteins 7:99-111; Pedersen (1985) Environ. Health Perspect. 61:185-190; and Kini et al. (1991) J. Biomol. Struct. Dyn. 9:475-488).

The SPL activity of an SPL polypeptide or variant thereof may generally be assessed using an in vitro assay that detects the degradation of labeled substrate (i.e., sphingosine-1-phosphate, or a derivative thereof). Within such assays, pyridoxal 5′-phosphate is a requirement for SPL activity. In addition, the reaction generally proceeds optimally at pH 7.4-7.6 and requires chelators due to sensitivity toward heavy metal ions. The substrate should be a D-erythro isomer, but in derivatives of sphingosine-1-phosphate the type and chain length of sphingoid base may vary. In general, an assay as described by Van Veldhoveet al., J Biol Chem, 1991; 266: 12502-07 may be employed. Briefly, a solution (e.g., a cellular extract) containing the polypeptide may be incubated with 40 μM radiolabeled or fluorescently labeled substrate at 37° C. for 1 hour in the presence of, for example, 50 mM sucrose, 100 mM K-phosphate buffer pH 7.4, 25 mM NaF, 0.1% (w/v) Triton X-100, 0.5 mM EDTA, 2 mM DTT, 0.25 mM pyridoxal phosphate. Reactions may then be terminated and analyzed by thin-layer chromatography to detect the formation of labeled fatty aldehydes and further metabolites. In general, a polypeptide has SPL activity if, within such an assay: (1) the presence of 2-50 μg polypeptide (or 0.1-10 mg/mL) results in a statistically significant increase in the level of substrate degradation, preferably a two-fold increase, relative to the level observed in the absence of polypeptide; and/or (2) the increase in the level of substrate degradation is pyridoxal 5′-phosphate dependent.

Within certain embodiments, an in vitro assay for SPL activity may be performed using cellular extracts prepared from cells that express the polypeptide of interest. Preferably, in the absence of a gene encoding an SPL polypeptide, such cells do not produce a significant amount of endogenous SPL (i.e., a cellular extract should not contain a detectable increase in the level of SPL, as compared to buffer alone without extract). It has been found, within the context of the present invention, that yeast cells containing deletion of the SPL gene (BST1) are suitable for use in evaluating the SPL activity of a polypeptide. bst1Δ cells can be generated from S. cerevisiae using standard techniques, such as PCR, as described herein. A polypeptide to be tested for SPL activity may then be expressed in bst1Δ cells, and the level of SPL activity in an extract containing the polypeptide may be compared to that of an extract prepared from cells that do not express the polypeptide. For such a test, a polypeptide is preferably expressed on a high-copy yeast vector (such as pYES2, which is available from Invitrogen) yielding more than 20 copies of the gene per cell. In general, a polypeptide has SPL activity if, when expressed using such a vector in a bst1Δ cell, a cellular extract results in a two-fold increase in substrate degradation over the level observed for an extract prepared from cells not expressing the polypeptide.

An additional assay for SPL enzyme activity is a fluorescence assay as described in Bandjuvula P., et al. 2007 J. Lipid Research: A Rapid Fluorescence Assay for Sphingosine-1-phosphate Lyase Enzyme Activity. This assay uses a commercially available ω(7-nitro-2-1,3-benzoxadiazol-4-yl)-D-erythro (NBD)-labeled fluorescent substrate. Enzyme activity is determined by following the formation of NBD-aldehyde product, which is isolated from unreacted substrate by lipid extraction and quantified after separation by HPLC using a C18 column. This method is suitable for quantifying SPL activity in a variety of cell and tissue sources.

A further test for SPL activity may be based upon functional complementation in the bst1Δ strain. It has been found, within the context of the present invention, that bst1Δ cells are highly sensitive to D-erythro-sphingosine. In particular, concentrations as low as 10 μM sphingosine completely inhibit the growth of bst1Δ cells. Such a level of sphingosine has no effect on the growth of wildtype cells. A polypeptide having SPL activity as provided above significantly diminishes (i.e., by at least two fold) the sphingosine sensitivity when expressed on a high-copy yeast vector yielding more than 20 copies of the gene per cell.

Sphingolipid measurements may be performed using a variety of assays known in the art. For example, sphingolipids such as S1P may be measured by isolating lipids, derivatizing them with ortho-phthalaldehyde, separation on HPLC and detection by fluorescence detection as described in Bandhuvula P, et al., J Biol Chem 2005; 280: 33697-33700.

In general, SPL polypeptides, and polynucleotides encoding such polypeptides, may be prepared using any of a variety of techniques that are well known in the art. For example, a DNA sequence encoding native SPL may be prepared by amplification from a suitable cDNA or genomic library using, for example, polymerase chain reaction (PCR) or hybridization techniques. Libraries may generally be prepared and screened using methods well known to those of ordinary skill in the art, such as those described in Sambrook et al., Molecular Cloning: A Laboratory Manual, Cold Spring Harbor Laboratories, Cold Spring Harbor, N.Y., 1989. cDNA libraries may be prepared from any of a variety of sources known to contain enzymes having SPL activity. SPL activity is ubiquitous with regard to species and mammalian tissues, with the exception of platelets, in which SPL activity is notably absent. In rat tissues, the highest levels of activity have been demonstrated in intestinal mucosa, liver and Harderian gland, with low activity in skeletal muscle and heart. Activity has also been demonstrated in a number of human (hepatoma cell line HB 8065, cervical carcinoma HeLa), mouse (hepatoma line BW1, mouse embryo 3T3-L1, Swiss 3T3 cells) and other cell lines, as well as in human cultured fibroblasts. Preferred cDNA libraries may prepared from human liver, intestine or brain tissues or cells. Other libraries that may be employed will be apparent to those of ordinary skill in the art. Primers for use in amplification may be readily designed based on the sequence of a native SPL polynucleotide, as provided herein in SEQ ID NOs:1, 3, 5, 7, 9, 12, 15, and 17.

Alternatively, an endogenous SPL gene may be identified using a screen for cDNAs that complement the BST1 deletion in yeast. A cDNA expression library may be generated using a regulatable yeast expression vector (e.g., pYES, which is available from Invitrogen, Inc.) and standard techniques. A yeast bst1Δ strain may then be transformed with the cDNA library, and endogenous cDNAs having the ability to functionally complement the yeast lyase defect (i.e., restore the ability to grow in the presence of D-erythro-sphingosine) may be isolated.

An endogenous SPL gene may also be identified based on cross-reactivity of the protein product with anti-SPL antibodies, which may be prepared as described herein. Such screens may generally be performed using standard techniques (see Huynh et al., Construction and Screening cDNA Libraries in λgt11, in D M Glover, ed., DNA Cloning: A Practical Approach, 1984; 1: 49-78, IRL Press, Oxford).

Polynucleotides encompassed by the present invention include DNA and RNA molecules that encode an endogenous SPL polypeptide. Such polynucleotides include those that comprise a sequence recited in any one of SEQ ID NOs:1, 3, 5, 7, 9, 12, 15, and 17. Also encompassed are other polynucleotides that encode an SPL amino acid sequence encoded by such polynucleotides, as well as polynucleotides that encode variants of a native SPL sequence that retain SPL activity. Polynucleotides encompassed by the present invention include polynucleotides having at least about 70%, 75%, 80%, 85%, 90%, 91%, 92%, 93%, 94%, 95%, 96%, 97%, 98%, or 99% or more identity along its length, to an SPL polynucleotide sequence set forth herein. Polynucleotides that are substantially homologous to a sequence complementary to an endogenous SPL gene are also within the scope of the present invention. “Substantial homology,” as used herein refers to polynucleotides that are capable of hybridizing under moderately stringent conditions to a polynucleotide complementary to an SPL polynucleotide sequence provided herein, provided that the encoded SPL polypeptide variant retains SPL activity. Suitable moderately stringent conditions include prewashing in a solution of 5×SSC, 0.5% SDS, 1.0 mM EDTA (pH 8.0); hybridizing at 50-65° C., 5×SSC, overnight; followed by washing twice at 65° C. for 20 minutes with each of 2×, 0.5× and 0.2×SSC containing 0.1% SDS. Nucleotide sequences that, because of code degeneracy, encode a polypeptide encoded by any of the above sequences are also encompassed by the present invention.

Polypeptides of the present invention may be prepared by expression of recombinant DNA encoding the polypeptide in cultured host cells. Preferably, the host cells are bacteria, yeast, insect or mammalian cells, and more preferably the host cells are S. cerevisiae bst1Δ cells. The recombinant DNA may be cloned into any expression vector suitable for use within the host cell and transfected into the host cell using techniques well known to those of ordinary skill in the art. In certain embodiments, adenoviral expression vectors can be used to express SPL polypeptides in mammalian host cells such as any of a variety of human host cells (e.g., HEK293 or other cells). A suitable expression vector contains a promoter sequence that is active in the host cell. A tissue-specific or conditionally active promoter may also be used. Preferred promoters express the polypeptide at high levels.

Optionally, the construct may contain an enhancer, a transcription terminator, a poly(A) signal sequence, a bacterial or mammalian origin of replication and/or a selectable marker, all of which are well known in the art. Enhancer sequences may be included as part of the promoter region or separately. Transcription terminators are sequences that stop RNA polymerase-mediated transcription. The poly(A) signal may be contained within the termination sequence or incorporated separately. A selectable marker includes any gene that confers a phenotype on the host cell that allows transformed cells to be identified. Such markers may confer a growth advantage under specified conditions. Suitable selectable markers for bacteria are well known and include resistance genes for ampicillin, kanamycin and tetracycline. Suitable selectable markers for mammalian cells include hygromycin, neomycin, genes that complement a deficiency in the host (e.g., thymidine kinase and TK-cells) and others well known in the art. For yeast cells, one suitable selectable marker is URA3, which confers the ability to grow on medium without uracil.

Polynucleotide sequences expressed in this manner may encode a native SPL polypeptide (e.g., human), or may encode portions or other variants of native SPL polypeptide. Polynucleotide sequences encoding variants of a native SPL may generally be prepared using standard mutagenesis techniques, such as oligonucleotide-directed site-specific mutagenesis, and sections of the polynucleotide sequence may be removed to permit preparation of truncated polypeptides.

To generate cells that express a polynucleotide encoding an SPL polypeptide, cells may be transfected, transformed or transduced using any of a variety of techniques known in the art. Any number of transfection, transformation, and transduction protocols known to those in the art may be used, for example those outlined in Current Protocols in Molecular Biology, John Wiley & Sons, New York. N.Y., or in numerous kits available commercially (e.g., Invitrogen Life Technologies, Carlsbad, Calif.). Such techniques may result in stable transformants or may be transient. One suitable transfection technique is electroporation, which may be performed on a variety of cell types, including mammalian cells, yeast cells and bacteria, using commercially available equipment. Optimal conditions for electroporation (including voltage, resistance and pulse length) are experimentally determined for the particular host cell type, and general guidelines for optimizing electroporation may be obtained from manufacturers. Other suitable methods for transfection will depend upon the type of cell used (e.g., the lithium acetate method for yeast), and will be apparent to those of ordinary skill in the art. Following transfection, cells may be maintained in conditions that promote expression of the polynucleotide within the cell. Appropriate conditions depend upon the expression system and cell type, and will be apparent to those skilled in the art.

SPL polypeptides may be expressed in transfected cells by culturing the cell under conditions promoting expression of the transfected polynucleotide. Appropriate conditions will depend on the specific host cell and expression vector employed, and will be readily apparent to those of ordinary skill in the art. For commercially available expression vectors, the polypeptide may generally be expressed according to the manufacturer's instructions. For certain purposes, expressed polypeptides of this invention may be isolated in substantially pure form. Preferably, the polypeptides are isolated to a purity of at least 80% by weight, more preferably to a purity of at least 95% by weight, and most preferably to a purity of at least 99% by weight. In general, such purification may be achieved using, for example, the standard techniques of ammonium sulfate fractionation, SDS-PAGE electrophoresis, and/or affinity chromatography.

According to certain related embodiments, an agent that causes a decreased SPL expression level may be an antisense polynucleotide that specifically hybridizes to a nucleic acid molecule that encodes a SPL polypeptide, a ribozyme that specifically cleaves a nucleic acid molecule that encodes a SPL polypeptide, a small interfering RNA that is capable of interfering with a nucleic acid molecule that encodes a SPL polypeptide, or an agent that alters activity of a regulatory element that is operably linked to a nucleic acid molecule that encodes a SPL polypeptide. As disclosed herein and known to the art, such nucleic acid sequence-based agents can be readily prepared using routine methodologies

A polynucleotide that is complementary to at least a portion of a coding sequence (e.g., an antisense polynucleotide or a ribozyme) may thus be used to modulate SPL-encoding gene expression. Identification of oligonucleotides and ribozymes for use as antisense agents, and DNA encoding genes for their targeted delivery, involve methods well known in the art. For example, the desirable properties, lengths and other characteristics of such oligonucleotides are well known. Antisense oligonucleotides are typically designed to resist degradation by endogenous nucleolytic enzymes by using such linkages as: phosphorothioate, methylphosphonate, sulfone, sulfate, ketyl, phosphorodithioate, phosphoramidate, phosphate esters, and other such linkages (see, e.g., Agrwal et al., Tetrahedron Lett 1987; 28: 3539-3542; Miller et al., J Am Chem Soc 1971; 93: 6657-6665; Stec et al., Tetrahedron Lett. 1971; 26: 2191-2194; Moody et al., Nucl Acids Res. 1989; 12: 4769-4782; Uznanski et al., Nucl Acids Res 1989; Letsinger et al., Tetrahedron 1984; 40: 137-143; Eckstein, Annu Rev Biochem 1985; 54: 367-402; Eckstein, Trends Biol Sci 1989; 14: 97-100; Stein In: Oligodeoxynucleotides, Antisense Inhibitors of Gene Expression, Cohen, Ed, Macmillan Press, London, pp. 97-117, 1989; Jager et al., Biochemistry 1988; 27: 7237-7246).

Antisense polynucleotides are oligonucleotides that bind in a sequence-specific manner to nucleic acids, such as mRNA or DNA. When bound to mRNA that has complementary sequences, antisense prevents translation of the mRNA (see, e.g., U.S. Pat. No. 5,168,053 to Altman et al.; U.S. Pat. No. 5,190,931 to Inouye, U.S. Pat. No. 5,135,917 to Burch; U.S. Pat. No. 5,087,617 to Smith; and Clusel et al., Nucl Acids Res 1993; 21: 3405-3411, which describes dumbbell antisense oligonucleotides). Triplex molecules refer to single DNA strands that bind duplex DNA forming a colinear triplex molecule, thereby preventing transcription (see, e.g., U.S. Pat. No. 5,176,996 to Hogan et al., which describes methods for making synthetic oligonucleotides that bind to target sites on duplex DNA).

Particularly useful antisense nucleotides and triplex molecules are molecules that are complementary to or bind the sense strand of DNA or mRNA that encodes a SPL polypeptide or a protein mediating any other process related to expression of endogenous SPL, such that inhibition of translation of mRNA encoding the SPL polypeptide is effected. cDNA constructs that can be transcribed into antisense RNA may also be introduced into cells or tissues to facilitate the production of antisense RNA. Antisense technology can be used to control gene expression through interference with binding of polymerases, transcription factors or other regulatory molecules (see Gee et al., in Huber and Carr, Molecular and Immunologic Approaches, Futura Publishing Co., Mt. Kisco, N.Y., 1994). Alternatively, an antisense molecule may be designed to hybridize with a control region of a SPL-encoding gene (e.g., promoter, enhancer or transcription initiation site), and block transcription of the gene; or to block translation by inhibiting binding of a transcript to ribosomes.

According to certain embodiments the present invention also contemplates use of SPL-encoding nucleic acid sequence-specific ribozymes. A ribozyme is an RNA molecule that specifically cleaves RNA substrates, such as mRNA, resulting in specific inhibition or interference with cellular gene expression. There are at least five known classes of ribozymes involved in the cleavage and/or ligation of RNA chains. Ribozymes can be specifically targeted to any RNA transcript and can catalytically cleave such transcripts (see, e.g., U.S. Pat. No. 5,272,262; U.S. Pat. No. 5,144,019; and U.S. Pat. Nos. 5,168,053, 5,180,818, 5,116,742 and 5,093,246 to Cech et al.). Any SPL mRNA-specific ribozyme, or a nucleic acid encoding such a ribozyme, may be delivered to a host cell to effect inhibition of SPL gene expression. Ribozymes may therefore be delivered to the host cells by DNA encoding the ribozyme linked to a eukaryotic promoter, such as a eukaryotic viral promoter, such that upon introduction into the nucleus, the ribozyme will be directly transcribed. Particularly useful sequence regions of a SPL-encoding mRNA for use as a ribozyme target can be found using routine sequence alignment tools known to the art such as BLAST or MegAlign, and may preferably be sequence stretches that are unique to the SPL-encoding mRNA relative to other transcribed sequences that may be present in a particular cell. In one preferred example, a ribozyme may be designed and constructed such that it is targeted to bind to a region of SPL-encoding mRNA that encodes a portion of the human SPL polypeptide containing the lysine residue found at amino acid position 353 therein, but the invention is not so limited and other ribozymes targeted to other regions of SPL-encoding mRNA are also contemplated.

Any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine and wybutosine, as well as acetyl-methyl-, thio- and other modified forms of adenine, cytidine, guanine, thymine and uridine.

Without being bound by theory, it is thought that SPL is degraded by a protease. As such, site-directed mutagenesis that alters the protease cleavage site can be used to alter the activity of the SPL protein. In certain embodiments, the site can be mutated to increase cleavage, which would destabilize the protein. In other embodiments, the cleavage site can be mutated such that cleavage is decreased, this stabilizing the SPL. Site-directed mutagenesis can be carried out using techniques known in the art, such as those described Current Protocols in Molecular Biology, John Wiley & Sons, New York. N.Y.

RNA interference (RNAi) is a polynucleotide sequence-specific, post-transcriptional gene silencing mechanism effected by double-stranded RNA that results in degradation of a specific messenger RNA (mRNA), thereby reducing the expression of a desired target polypeptide encoded by the mRNA (see, e.g., WO 99/32619; WO 01/75164; U.S. Pat. No. 6,506,559; Fire et al., Nature 1998; 391: 806-11; Sharp, Genes Dev. 1999; 13: 139-41; Elbashir et al., Nature 2001; 411: 494-98; Harborth et al., J Cell Sci. 2001; 114: 4557-65). “Small interfering RNA” (siRNA) or DNP-RNA polynucleotides that interfere with expression of specific polypeptides in higher eukaryotes such as mammals (including humans) have been considered (e.g., Karagiannis et al., Cancer Gene Ther, May 2005, PMID: 15891770; Chen et al., Drug Discov Today 2005; 10: 587; Scherr et al., Curr Opin Drug Discov Devel 2005; 8: 262; Tomari et al., Genes Dev 2005; 19: 517; see also, e.g., Tuschl, Chembiochem 2001; 2: 239-245; Sharp, Genes Dev 2001; 15: 485; Bernstein et al., RNA 2001; 7: 1509; Zamore, Science 2002; 296: 1265; Plasterk, Science 2002; 296: 1263; Zamore Nat Struct Biol 2001; 8: 746; Matzke et al., Science 2001; 293: 1080; Scadden et al., EMBO Rep 2001; 2: 1107; Hutvagner et al., Curr Opin Gen Dev 2002; 12: 225-32; Elbashir et al., 2001; Nykanen et al., Cell 2001; 107: 309-21; Bass, Cell 2000; 101: 235-38; Zamore et al., Cell 2000; 101: 25-33). Transfection of human and other mammalian cells with double-stranded RNAs of about 18-27 nucleotide base pairs in length interferes in a sequence-specific manner with expression of particular polypeptides encoded by messenger RNAs (mRNA) containing corresponding nucleotide sequences (WO 01/75164; Elbashir et al., 2001; Elbashir et al., Genes Dev. 2001; 15: 188-200); Harborth et al., J Cell Sci 2001; 114: 4557-65; Carthew et al., Curr Opin Cell Biol 2001; 13: 244-48; Mailand et al., Nature Cell Biol, Advance Online Publication Mar. 18, 2002; Mailand et al., Nature Cell Biol 2002; 4: 317). SPL-specific siRNA constructs are available and have been obtained from Dharmacon (Lafayette, Colo.). In certain non-limiting embodiments double-stranded RNAs for use in RNAi may have, for example, 17, 18, 19, 20, 21, 22, 23, 24, 25, 26, 27, 28, 29, 30, 31, 32 or more nucleotide base pairs.

As noted above, in certain embodiments the agent that causes a decreased SPL expression level may alter activity of a regulatory element that is operably linked to a nucleic acid molecule that encodes a SPL polypeptide. According to a related embodiment the regulatory element comprises a GATA transcription factor-binding motif (Oskouian et al., J Biol Chem 2005; 280: 18403-410). By way of representative example and not limitation, these and related embodiments contemplate suitable agents that are capable of down-regulating SPL activity by suppressing or repressing transcription of SPL-encoding genes, which agents can be readily identified using art-accepted methodologies to screen for functional blockers of SPL gene transcription. For instance, Oskouian et al. (2005) describe regulation of the human SPL gene by a GATA transcription factor that interacts with an upstream regulatory element, as characterized using a reporter gene transcriptional run-off assay that can be readily adapted to screen for functional inhibitors of SPL gene expression. Using such an approach, agents capable of causing decreased SPL expression levels may be identified from compound libraries such as libraries of synthetic (e.g., combinatorial chemistry) small molecules, or natural products libraries, or recombinant expression libraries, or other sources. Agents of the present invention that inhibit SPL may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. New potential therapeutic agents may also be created using methods such as rational drug design or computer modelling.

Certain other embodiments as disclosed herein contemplate a method for preventing or treating cardiac, stroke, or ischemia/reperfusion injury in a subject that comprises administering an agent that alters sphingosine-1-phosphate lyase (SPL) activity, wherein the agent is selected from (a) a mutated form of a nucleic acid molecule that encodes a SPL polypeptide wherein the mutated form encodes a dominant negative mutant SPL polypeptide, or a complementary polynucleotide thereto, and (b) a dominant negative mutant SPL polypeptide encoded by (a). The nucleic acid molecule that encodes a SPL polypeptide is described above and in several above-referenced patents and patent applications, e.g., U.S. application Ser. No. 10/348,052, U.S. application Ser. No. 10/622,011, and PCT/US2003/01739, including in publications cited therein (e.g., Van Veldhoven et al., J Biol Chem 1991; 266: 12502-07), as are compositions and methods for introducing mutations (see, e.g., Sambrook et al., 1989; Ausubel et al., 1993; Maniatis et al., 1982). Principles and practices directed to functional inactivation of a desired target gene using a dominant negative mutation are described in Herskowitz et al. (Nature 1987; 329: 219-222) and in Perlmutter et al. (Curr Opin Immunol 1996; 8: 285-290). Without wishing to be bound by theory, according to these and related embodiments a dominant negative mutant SPL may be engineered and functionally identified using routine methodologies as described herein and in the cited references, such that introduction of the dominant negative mutant (e.g., by administering a mutated SPL-encoding nucleic acid molecule by suitable art-accepted methodologies such as transfection, electroporation, biolistics, naked DNA, plasmid, viral vector, liposomal delivery or other suitable means, or by administering a mutant SPL polypeptide) in a manner and for a time sufficient to obtain a cell having the dominant negative SPL, results in competition for substrate (e.g., S1P) between wildtype (endogenous) SPL and the dominant negative mutant SPL, thereby effectively decreasing the effective level of SPL activity in the cell.

Certain other embodiments described herein relate to antibodies that bind to an SPL polypeptide. Antibodies may function as inhibiting agents (as discussed further below) to inhibit or block SPL activity in vivo or in vitro, including in cells or tissues ex vivo which may in certain embodiments then be reintroduced to an in vivo environment, such as in a subject in whom therapeutic benefit may be desired. Alternatively, or in addition, antibodies may be used within screening assays for endogenous SPL polypeptides or for inhibiting agents, for purification of SPL polypeptides, for assaying the level of SPL within a sample and/or for studies of SPL expression. Such antibodies may be polyclonal or monoclonal, and are generally specific for one or more SPL polypeptides and/or one or more variants thereof.

Antibodies may be prepared by any of a variety of techniques known to those of ordinary skill in the art (see, e.g., Harlow et al., Antibodies: A Laboratory Manual, Cold Spring Harbor Laboratory, 1988). In one such technique, an immunogen comprising an SPL polypeptide or antigenic portion thereof is initially injected into a suitable animal (e.g., mice, rats, rabbits, sheep and goats), preferably according to a predetermined schedule incorporating one or more booster immunizations. To increase immunogenicity, an immunogen may be linked to a carrier, for example, via glutaraldehyde coupling to albumin or keyhole limpet hemocyanin (KLH). Following injection, the animals are bled periodically to obtain post-immune serum containing polyclonal anti-SPL antibodies. Polyclonal antibodies may then be purified from such antisera by, for example, affinity chromatography using an SPL polypeptide or antigenic portion thereof coupled to a suitable solid support. Such polyclonal antibodies may be used directly for screening purposes and for Western blots.

For instance, an adult rabbit (e.g., NZW) may be immunized with 10 μg purified (e.g., using a nickel-column) His-tagged recombinant human SPL polypeptide emulsified in complete Freund's adjuvant (1:1 v/v) in a volume of 1 mL. Immunization may be achieved via injection in at least six different subcutaneous sites. For subsequent immunizations, 5 μg of an SPL polypeptide may be emulsified in complete Freund's adjuvant and injected in the same manner. Immunizations may continue until a suitable serum antibody titer is achieved (typically a total of about three immunizations). The rabbit may be bled immediately before immunization to obtain pre-immune serum, and then 7-10 days following each immunization.

For certain embodiments, monoclonal antibodies may be desired. Monoclonal antibodies may be prepared, for example, using the technique of Kohler et al., Eur J Immunol 1976; 6:511-519, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction.

Accordingly, in certain contemplated embodiments antibodies may be, for example, polyclonal, monoclonal, single chain, chimeric, humanized, anti-idiotypic, or CDR-grafted immunoglobulins, or antigen-binding (e.g., SPL-binding) fragments thereof, such as proteolytically generated or recombinantly produced immunoglobulin F(ab′)₂, Fab, Fab′, Fv, and/or Fd fragments, single domain antibodies (“dAbs”; Holt et al., 2003 Trends Biotech. 21:484) and diabodies (Hudson et al., 1999 J. Immunol. Meth. 231:177). An antibody according to the present invention may belong to any immunoglobulin class, for example IgG, IgE, IgM, IgD, or IgA. It may be obtained from or derived from an animal, for example, fowl (e.g., chicken) or a mammal, which includes but is not limited to a mouse, rat, hamster, rabbit, or other rodent, a cow, horse, sheep, goat, camel, human or other primate. The antibody may be an internalizing antibody, or the antibody may be modified so that it may be easily transported across a cell membrane.

Certain preferred antibodies are those antibodies that inhibit SPL activity, for instance, by blocking SPL from interacting with its cognate specific substrate(s) and/or co-factors, which may be, for example and according to non-limiting theory, by direct antibody blockade of an interaction site, by antibody steric hindrance of such interactions, by antibody-induced conformational change of SPL in a manner that precludes SPL function, or by another mechanism. Binding properties of an antibody to SPL may generally be assessed using conventional immunodetection methods including, for example, an enzyme-linked immunosorbent assay (ELISA), immunoprecipitation, radioimmunoassay (RIA), immunoblotting (e.g., western blot) and the like, which may be readily performed by those having ordinary skill in the art.

Methods well known in the art and described herein may be used to generate antibodies, including polyclonal antisera or monoclonal antibodies, that are specific for SPL as may be desired. Antibodies also may be produced as genetically engineered immunoglobulins (Ig) or Ig fragments designed to have desirable properties. For example, by way of illustration and not limitation, antibodies may include a recombinant IgG that is a chimeric fusion protein having at least one variable (V) region domain from a first mammalian species and at least one constant region domain from a second, distinct mammalian species (see, e.g., Morrison et al., Proc. Natl. Acad. Sci. USA, 81:6851-55 (1984); Shin et al., Methods Enzymol. 178:459-76 (1989); Walls et al., Nucleic Acids Res. 21:2921-29 (1993); U.S. Pat. No. 5,482,856). Most commonly, a chimeric antibody has murine variable region sequences and human constant region sequences. Such a murine/human chimeric immunoglobulin may be “humanized” by grafting the complementarity determining regions (CDRs) derived from a murine antibody, which confer binding specificity for an antigen, into human-derived V region framework regions and human-derived constant regions (see, e.g., Jones et al., Nature 321:522-25 (1986); Riechmann et al., Nature 332:323-27 (1988); Padlan et al., FASEB 9:133-39 (1995); Chothia et al., Nature, 342:377-383 (1989); Bajorath et al., Ther. Immunol. 2:95-103 (1995); EP-0578515-A3). Fragments of these molecules may be generated by proteolytic digestion, or optionally, by proteolytic digestion followed by mild reduction of disulfide bonds and alkylation. Alternatively, such fragments may also be generated by recombinant genetic engineering techniques (e.g., Harris, W. J., Adair, J. R., (Eds.) 1997 Antibody Therapeutics, CRC Press, Boca Raton, Fla.).

An antibody that is immunospecific or that specifically binds to a SPL polypeptide as provided herein reacts at a detectable level with the SPL and not with molecules having distinct or unrelated structures, preferably with an affinity constant, Kₐ, of greater than or equal to about 10⁴ M⁻¹, more preferably of greater than or equal to about 10⁵ M⁻¹, more preferably of greater than or equal to about 10⁶ M⁻¹, and still more preferably of greater than or equal to about 10⁷ M⁻¹. Affinity of an antibody for its cognate antigen is also commonly expressed as a dissociation constant K_{D}, and an anti-SPL antibody specifically binds to the SPL if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M. Affinities of binding partners or antibodies can be readily determined using conventional techniques, for example, those described by Scatchard et al. (Ann. N.Y. Acad. Sci. USA 51:660 (1949)) or by surface plasmon resonance (BIAcore, Biosensor, Piscataway, N.J.). See, e.g., Wolff et al., Cancer Res. 53:2560-2565 (1993).

Antibodies may generally be prepared by any of a variety of techniques known to those skilled in the art. See, e.g., Harlow et al., Antibodies: A Laboratory Manual, Cold Spring Harbor Laboratory (1988). In one such technique, an animal is immunized with an immunogenic form of the drug, for instance, using the drug as a hapten on a suitable carrier according to established methodologies, as an antigen to generate polyclonal antisera. Suitable animals include, for example, rabbits, sheep, goats, pigs, cattle, and may also include smaller mammalian species, such as mice, rats, and hamsters, or other species.

An immunogen may comprise a purified or partially purified SPL-derived peptide fragment or full-length SPL polypeptide, which may be generated using standard recombinant genetic methodologies, or by proteolytic cleavage of naturally occurring SPL proteins, or may be chemically synthesized. SPL polypeptides may be isolated by techniques known in the art such as polyacrylamide gel electrophoresis or any of a variety of other separation methods such as liquid chromatography or other suitable methodologies.

For raising antibodies to SPL polypeptides or peptides, peptides useful as immunogens typically may have an amino acid sequence of at least 4 or 5 consecutive amino acids from the SPL sequence, and preferably have at least 6, 7, 8, 9, 10, 11, 12, 14, 15, 16, 18, 19 or 20 consecutive amino acids of the SPL polypeptide sequence. Certain other preferred SPL peptide immunogens may comprise 21-25, 26-30, 31-35, 36-40, 41-50 or more consecutive amino acids of a SPL polypeptide sequence. SPL polypeptides or peptides useful for immunization may also be selected by analyzing the primary, secondary, and tertiary structure of the SPL polypeptide according to methods known to those skilled in the art, in order to determine amino acid sequences more likely to generate an antigenic response in a host animal. See, e.g., Novotny, 1991 Mol. Immunol. 28:201-207; Berzofsky, 1985 Science 229:932-40; Chang et al. J. Biochem. 117:863-68 (1995); Kolaskar et al. Viology 261:31-42 (1999)). Preferably, the SPL polypeptide or peptide comprises a sufficient number of amino acids to fold in a manner that approximates the conformation of the SPL polypeptide in its physiologically active form.

Immunogens may be prepared and animals immunized according to methods well known in the art. See, e.g., Harlow et al., Antibodies: A Laboratory Manual, Cold Spring Harbor Laboratory (1988). The immune response may be monitored by periodically bleeding the animal, separating the sera out of the collected blood, and analyzing the sera in an immunoassay, such as an ELISA or Ouchterlony diffusion assay, or the like, to determine the specific antibody titer. Once an antibody titer is established, the animals may be bled periodically to accumulate the polyclonal antisera. Polyclonal antibodies that bind specifically to the drug may then be purified from such antisera, for example, by affinity chromatography using S. aureus protein A or protein G, which specifically binds to a constant region (heavy or light chain) of the antibody(ies) to be purified, or using the drug, immobilized on a suitable solid support.

Monoclonal antibodies that specifically bind to SPL, and hybridomas, which are immortal eukaryotic cell lines, that produce monoclonal antibodies having the desired binding specificity, may also be prepared, for example, using the technique of Kohler and Milstein (Nature, 256:495-497; 1976, Eur. J. Immunol. 6:511-519 (1975)) and improvements thereto with which a skilled artisan will be familiar. An animal—for example, a rat, hamster, or a mouse—is immunized with a SPL polypeptide or peptide immunogen; lymphoid cells that include antibody-forming cells, typically spleen cells, are obtained from the immunized animal; and such cells may be immortalized by fusion with a selection agent-sensitized myeloma (e.g., plasmacytoma) cell fusion partner.

Monoclonal antibodies may be isolated from the supernatants of hybridoma cultures or isolated from a mouse that has been treated (e.g., pristane-primed) to promote formation of ascites fluid containing the monoclonal antibody. Antibodies may be purified by affinity chromatography using an appropriate ligand selected based on particular properties of the monoclonal antibody (e.g., heavy or light chain isotype, binding specificity, etc.). Examples of a suitable ligand, immobilized on a solid support, include Protein A, Protein G, an anti-constant region (light chain or heavy chain) antibody, an anti-idiotype antibody and the drug antigen for which specific antibodies are desired.

Human monoclonal antibodies may be generated by any number of techniques with which those having ordinary skill in the art will be familiar. Antibodies may also be identified and isolated from human immunoglobulin phage libraries, from rabbit immunoglobulin phage libraries, and/or from chicken immunoglobulin phage libraries (see, e.g., Winter et al., 1994 Annu. Rev. Immunol. 12:433-55; Burton et al., 1994 Adv. Immunol. 57:191-280; U.S. Pat. No. 5,223,409; Huse et al., 1989 Science 246:1275-81; Schlebusch et al., 1997 Hybridoma 16:47-52 and references cited therein; Rader et al., J. Biol. Chem. 275:13668-76 (2000); Popkov et al., J. Mol. Biol. 325:325-35 (2003); Andris-Widhopf et al., J. Immunol. Methods 242:159-31 (2000)), or by other methodologies such as ribosome display (e.g., Hanes et al., 1998 Proc. Nat. Acad. Sci. USA 95:14130) or yeast display (e.g., Colby et al., 2004 Meths. Enzymol. 388:348) or the like. Antibodies isolated from non-human species or non-human immunoglobulin libraries may be genetically engineered according to methods described herein and known in the art to “humanize” the antibody or fragment thereof.

In certain embodiments, a B cell from an immunized animal that is producing an anti-SPL antibody is selected and the light chain and heavy chain variable regions are cloned from the B cell according to molecular biology techniques known in the art (WO 92/02551; U.S. Pat. No. 5,627,052; Babcook et al., Proc. Natl. Acad. Sci. USA 93:7843-48 (1996)) and described herein. Preferably B cells from an immunized animal are isolated from the spleen, lymph node, or peripheral blood sample by selecting a cell that is producing an antibody that specifically binds to SPL. B cells may also be isolated from humans, for example, from a peripheral blood sample.

An antibody fragment may also be any synthetic or genetically engineered protein that acts like an antibody in that it binds to a specific antigen to form a complex. For example, antibody fragments include isolated fragments consisting of the light chain variable region; “Fv” fragments consisting of the variable regions of the heavy and light chains; recombinant single chain polypeptide molecules in which light and heavy variable regions are connected by a peptide linker (scFv proteins); and minimal recognition units consisting of the amino acid residues that mimic the hypervariable region. Such an antibody fragment preferably comprises at least one variable region domain. (see, e.g., Bird et al., Science 242:423-26 (1988); Huston et al., Proc. Natl. Acad. Sci. USA 85:5879-5883 (1988); EP-B1-0318554; U.S. Pat. No. 5,132,405; U.S. Pat. No. 5,091,513; and U.S. Pat. No. 5,476,786).

In certain embodiments, an antibody that specifically binds to a SPL may be an antibody that is expressed as an intracellular protein. Such intracellular antibodies are also referred to as intrabodies and may comprise a Fab fragment, or preferably comprise a scFv fragment (see, e.g., Lecerf et al., Proc. Natl. Acad. Sci. USA 98:4764-49 (2001)). The framework regions flanking the CDR regions can be modified to improve expression levels and solubility of an intrabody in an intracellular reducing environment (see, e.g., Worn et al., J. Biol. Chem. 275:2795-803 (2000)). An intrabody may be directed to a particular cellular location or organelle, for example by constructing a vector that comprises a polynucleotide sequence encoding the variable regions of an intrabody that may be operatively fused to a polynucleotide sequence that encodes a particular target antigen within the cell (see, e.g., Graus-Porta et al., Mol. Cell. Biol. 15:1182-91 (1995); Lener et al., Eur. J. Biochem. 267:1196-205 (2000)). An intrabody may be introduced into a cell by a variety of techniques available to the skilled artisan including via a gene therapy vector, or a lipid mixture (e.g., Provectin™ manufactured by Imgenex Corporation, San Diego, Calif.), or according to photochemical internalization methods.

To identify other SPL-inhibiting agents, any of a variety of screens may be performed. Candidate inhibiting agents may be obtained using well known techniques from a variety of sources, such as plants, fungi or libraries of chemicals, small molecules or random peptides. Antibodies that bind to an SPL polypeptide, and anti-sense polynucleotides that hybridize to a polynucleotides that encodes an SPL, may be candidate inhibiting agents. Preferably, an inhibiting agent has a minimum of side effects and is non-toxic. For some applications, agents that can penetrate cells are preferred.

In certain embodiments, rational drug design is used to identify suitable agents. For example, drug design based on conjugation to the SPL active site, competitive binding to a substrate or cofactor, or similarity to THI or metabolite of THI or other imidazole compounds, etc, can be used to design appropriate agents. In this regard, metabolites include but are not limited to glucuronidated, phosphorylated, and methylated THI products (see e.g. V. Sarvesh et al. 2001 Drug Metabolism and Disposition 29:1290; Huskey et al, 1994 Drug Metabolism and Disposition 22: 651). In certain embodiments, glucuronidation occurs either on the imidazole ring or one of the four hydroxy groups. In a further embodiment, a THI metabolite may comprise a carbonyl compound (e.g., a ketone or an aldehyde) resulting from oxidizing any of the hydroxy groups (e.g., via P450 catalyzed oxidation). In another embodiment, a metabolite of THI comprises an alcohol-containing compound resulting from the carbonyl reduction (e.g., by carbonyl reductases) of the ketone.

Screens for inhibiting agents that decrease SPL expression or stability may be readily performed using well known techniques that detect the level of SPL protein or mRNA. Suitable assays include RNAse protection assays, in situ hybridization, ELISAs, Northern blots and Western blots. Such assays may generally be performed using standard methods (see Sambrook et al., Molecular Cloning: A Laboratory Manual, Cold Spring Harbor Laboratories, Cold Spring Harbor, N.Y., 1989).

For example, to detect mRNA encoding SPL, a nucleic acid probe complementary to all or a portion of the SPL gene sequence may be employed in a Northern blot analysis of mRNA prepared from suitable cells. Alternatively, realt-time PCR can also be used to detect levels of mRNA encoding SPL (see Gibson et al., Genome Research 1996; 6: 995-1001, Heid et al., Genome Research 1996; 6: 986-994). The first-strand cDNA to be used in the quantitative real-time PCR is synthesized from 20 μg of total RNA that is first treated with DNase I (e.g., Amplification Grade, Gibco BRL Life Technology, Gaitherburg, Md.), using Superscript Reverse Transcriptase (RT) (e.g., Gibco BRL Life Technology, Gaitherburg, Md.). Real-time PCR is performed, for example, with a GeneAmp™ 5700 sequence detection system (PE Biosystems, Foster City, Calif.). The 5700 system uses SYBR™ green, a fluorescent dye that only intercalates into double stranded DNA, and a set of gene-specific forward and reverse primers. The increase in fluorescence is monitored during the whole amplification process. The optimal concentration of primers is determined using a checkerboard. The PCR reaction is performed in 25 μl volumes that include 2.5 μl of SYBR green buffer, 2 μl of cDNA template and 2.5 μl each of the forward and reverse primers for the SPL gene, or other gene of interest. The cDNAs used for RT reactions are diluted approximately 1:10 for each gene of interest and 1:100 for the β-actin control. In order to quantify the amount of specific cDNA (and hence initial mRNA) in the sample, a standard curve is generated for each run using the plasmid DNA containing the gene of interest. Standard curves are generated using the Ct values determined in the real-time PCR which are related to the initial cDNA concentration used in the assay. Standard dilution ranging from 20-2×10⁶ copies of the SPL gene or other gene of interest are used for this purpose. In addition, a standard curve is generated for β-actin ranging from 200 fg-2000 fg. This practice enables standardization of the initial RNA content of a sample to the amount of β-actin for comparison purposes. The mean copy number for each sample tested is normalized to a constant amount of β-actin, allowing the evaluation of the observed expression levels of SPL or other gene of interest.

To detect SPL protein, a reagent that binds to the protein (typically an antibody, as described herein) may be employed within an ELISA or Western assay. Following binding, a reporter group suitable for direct or indirect detection of the reagent is employed (i.e., the reporter group may be covalently bound to the reagent or may be bound to a second molecule, such as Protein A, Protein G, immunoglobulin or lectin, which is itself capable of binding to the reagent). Suitable reporter groups include, but are not limited to, enzymes (e.g., horseradish peroxidase), substrates, cofactors, inhibitors, dyes, radionuclides, luminescent groups, fluorescent groups and biotin. Such reporter groups may be used to directly or indirectly detect binding of the reagent to a sample component using standard methods known to those of ordinary skill in the art.

To use such assays for identifying an inhibiting agent, the level of SPL protein or mRNA may be evaluated in cells treated with one or more candidate inhibiting agents. An increase or decrease in SPL levels may be measured by evaluating the level of SPL mRNA and/or protein in the presence and absence of candidate inhibiting agent. For example, an antisense inhibiting agent may be evaluated by assaying the effect on SPL levels. Suitable cells for use in such assays include the breast cancer cell lines MCF-7 (ATCC Accession Number HTB-22) and MDA-MB-231 (ATCC Accession Number HTB-26). A candidate modulator may be tested by transfecting the cells with a polynucleotide encoding the candidate and evaluating the effect of expression of the polynucleotide on SPL levels. Alternatively, the cells may be contacted with a candidate modulator, typically in an amount ranging from about 10 nM to about 10 mM. A candidate that results in a statistically significant change in the level of SPL mRNA and/or protein is an inhibiting agent.

Alternatively, or in addition, a candidate inhibiting agent may be tested for the ability to inhibit or increase SPL activity, using an in vitro assay as described herein (see Van Veldhoven et al., J Biol Chem 1991; 266: 12502-07) that detects the degradation of labeled substrate (i.e., sphingosine-1-phosphate, or a derivative thereof). Briefly, for example, a solution (e.g., a cellular extract) containing an SPL polypeptide (e.g., 10 nM to about 10 mM) may be incubated with a candidate inhibiting agent (typically 1 nM to 10 mM, preferably 10 nM to 1 mM) and a substrate (e.g., 40 μM) at 37° C. for 1 hour in the presence of, for example, 50 mM sucrose, 100 mM K-phosphate buffer pH 7.4, 25 mM NaF, 0.1% (w/v) Triton X-100, 0.5 mM EDTA, 2 mM DTT, 0.25 mM pyridoxal phosphate. Reactions may then be terminated and analyzed by thin-layer chromatography to detect the formation of labeled fatty aldehydes and further metabolites. An inhibiting agent (e.g., an antibody) that increases SPL activity results in a statistically significant increase in the degradation of sphingosine-1-phosphate, relative to the level of degradation in the absence of inhibiting agent. Such inhibiting agents may be used to increase SPL activity in a cell culture or a mammal, as described below.

An inhibiting agent may in certain embodiments additionally comprise, or may be associated with, a targeting component that serves to direct the agent to a desired tissue or cell type. As used herein, a “targeting component” may be any substance (such as a compound or cell) that, when linked to a compound enhances the transport of the compound to a target tissue, thereby increasing the local concentration of the compound. Targeting components include antibodies or fragments thereof, receptors, ligands and other molecules that bind to cells of, or in the vicinity of, the target tissue. Known targeting components include hormones, antibodies against cell surface antigens, lectins, adhesion molecules, tumor cell surface binding ligands, steroids, cholesterol, lymphokines, fibrinolytic enzymes and other drugs and proteins that bind to a desired target site. In particular, anti-tumor antibodies and compounds that bind to an estrogen receptor may serve as targeting components. As described herein, an antibody employed in certain presently disclosed embodiments may be an intact (whole) molecule, a fragment thereof, or a functional equivalent thereof. Examples of antibody fragments are F(ab′)2, −Fab′, Fab and F[v] fragments, or other fragments described herein, which may be produced by conventional methods, or by genetic or protein engineering, or according to other methodologies such as those described herein and known to the art. Linkage of an antibody or other targeting component to the agent that inhibits SPL activity may be via any suitable covalent bond using standard techniques that are well known in the art. Such linkage is generally covalent and may be achieved, for example, by direct condensation or other reactions, or by way of bi- or multi-functional linkers.

In certain embodiments, SPL inhibition affords cardioprotection by providing “toxic preconditioning” through accumulation of S1P and thereby blocking apoptosis and possibly other forms of cell death of e.g., cardiomyocytes or endothelial cells. Thus, agents that inhibit SPL may be identified by measuring apoptotic cells. Cells that are suspected of undergoing apoptosis may be examined for morphological, permeability or other changes that are indicative of an apoptotic state. For example by way of illustration and not limitation, apoptosis in many cell types may cause altered morphological appearance such as plasma membrane blebbing, cell shape change, loss of substrate adhesion properties or other morphological changes that can be readily detected by a person having ordinary skill in the art, for example by using light microscopy. As another example, cells undergoing apoptosis may exhibit fragmentation and disintegration of chromosomes, which may be apparent by microscopy and/or through the use of DNA-specific or chromatin-specific dyes that are known in the art, including fluorescent dyes. Such cells may also exhibit altered plasma membrane permeability properties as may be readily detected through the use of vital dyes (e.g., propidium iodide, trypan blue) or by the detection of lactate dehydrogenase leakage into the extracellular milieu. Another readily practiced method for detecting apoptotic cells relates to detection of altered plasma membrane outer leaflet phospholipid composition in such cells, as determined, for instance, by quantification of phosphatidylserine exteriorization in the plasma membrane using detectably labeled annexin V (e.g., Fadok et al., J Immunol 1992; 148: 2207). These and other means for detecting apoptotic cells by morphologic criteria, altered plasma membrane permeability and related changes will be apparent to those familiar with the art.

Apoptosis may also be determined by an assay for induction of specific protease activity in any member of a family of apoptosis-activated proteases known as the caspases (see, e.g., Green et al., Science 1998; 281: 1309). Those having ordinary skill in the art will be readily familiar with methods for determining caspase activity, for example by determination of caspase-mediated cleavage of specifically recognized protein substrates. These substrates may include, for example, poly-(ADP-ribose) polymerase (PARP) or other naturally occurring or synthetic peptides and proteins cleaved by caspases that are known in the art (see, e.g., Ellerby et al., J Neurosci 1997; 17: 6165). The synthetic peptide Z-Tyr-Val-Ala-Asp-AFC (SEQ ID NO:10), wherein “Z” indicates a benzoyl carbonyl moiety and AFC indicates 7-amino-4-trifluoromethylcoumarin (Kluck et al., Science 1997; 275: 1132; Nicholson et al., Nature 1995; 376: 37), is one such substrate. Other non-limiting examples of substrates include nuclear proteins such as U1-70 kDa and DNA-PKcs (Rosen and Casciola-Rosen, J Cell Biochem 1997; 64: 50; Cohen, Biochem J 1997; 326: 1). Cellular apoptosis may also be detected by determination of cytochrome c that has escaped from mitochondria in apoptotic cells (e.g., Liu et al., Cell 1996; 86: 147). Such detection of cytochrome c may be performed spectrophotometrically, immunochemically or by other well established methods for determining the presence of a specific protein. Alternatively, apoptosis or necrosis of the cell, and/or modulation of the functioning of the cell cycle within the cell, may be detected using art-established criteria. (See, e.g., Ashkenazi et al., Science 1998; 281: 1305; Thornberry et al., Science 1998; 281: 1312; Evan et al., Science 1998; 281: 1317; Adams et al., Science 1998; 281: 1322; and references cited therein; see also, e.g., Wyllie, Nature 1980; 284: 555; Arends et al., Am J Pathol 1990; 136: 593.) Persons having ordinary skill in the art will readily appreciate that there may be other suitable techniques for quantifying apoptosis.

### Methods of Use

The agents that inhibit SPL activity as described herein are useful in a variety of disease settings. In particular, the agents described herein that inhibit SPL may be used for the prevention and/or treatment of cardiac, stroke, and ischemia/reperfusion injury in a variety of settings.

The compositions and methods provided herein may be used for preventing and treating cardiac injury. In this regard, cardiac injury may comprise injury due to acute ischemia/reperfusion, coronary obstruction, cardiac percutaneous intervention, coronary artery bypass surgery, cardiopulmonary bypass, heart transplant surgery, and non-cardiac surgery. The present invention provides compositions and methods for treating any cardiac failure resulting from cellular injury, from any apoptotic or necrotic process, including congestive heart failure from any number of infectious, toxic, genetic/inborn errors or other metabolic causes. In certain embodiments, the compositions and methods disclosed herein are useful for the prevention or treatment of cardiac injury due to repair of congenital cardiac malformations (congenital heart disease). In other embodiments, the compositions and methods described herein are used for the prevention or treatment of cardiac injury due closure of septal defects by percutaneous means or percutaneous mitral valve repair or mitral valvulotomy. In other embodiments, the compositions and methods of the present invention may be used for the treatment or prevention of any one or more of cardiac injury due to hypoxia, due to hypoxia with reperfusion or with reoxygenation, myocardial infarction, acute or chronic congestive heart failure, and myocarditis. The compositions and methods described herein may be used for the prevention or treatment of cardiac injury due to cardiotoxicity resulting from any of a variety of drugs or therapeutic regimens that may result in such toxicity to, e.g., cardiomyocytes, including but not limited to anthracyclines, trastuzumab, doxorubicin and radiation treatment. The compositions and methods described herein may be used for the prevention or treatment of cardiac injury that may occur in the course of, or following, heart transplantation, or from iron overload.

Myocardial infarction is caused by the obstruction of the blood flow through one or more coronary blood vessels. If the blockage is sustained, the resulting ischemia can cause irreparable damage to the heart muscle. Thus, the medical community has focused on ways to prevent or minimize such blockage. This has been achieved by pharmacologic (thrombolysis), mechanical (stenting), and/or surgical (coronary bypass) means. Although restoration of the blood flow to the affected tissue is the prerequisite for damage control in the myocardium, relieving the ischemia brings about, paradoxically, an added risk in that reperfusion itself causes damage to the heart. The involvement of apoptosis in ischemic injury has been examined in a number of tissues such as brain and kidney (Linnik M, et al., Evidence supporting a role for programmed cell death in focal cerebral ischemia in rats, Stroke 1993; p. 2002-2009; Heron A, et al., J Neurochem 1993; 61: 1973-1976; Schumer M, et al., Am J Pathol 1992; 140 831-838; Blomgren K, et al., Biochem Biophys Res Commun 2003; 304(3): 551-9). In the cardiovascular system, apoptosis has been observed in isolated rat cardiomyocytes subjected to hypoxia, in reperfused rabbit and rat hearts, and in myocardial autopsy tissue after death from acute myocardial infarction (Tanaka M, et al., Circ Res, 1994; 75: 426-433; Kajstura J, et al., Lab Invest, 1996; 74: 86-107; Gottlieb R, et al., J Clin Invest 1994; 94: 1621-1628; Buerke M, et al., Proc Natl Acad Sci USA 1995:92:8031-8035; Itoh G, et al., Am J Pathol 1995:146:1325-1331; Veinot J, et al., Hum Pathol 1997; 28: 485-492). The causes of apoptosis in ischemic or reperfused myocardium, however, remain obscure. Moreover, it is not yet clear whether myocardial apoptosis is triggered during ischemia and progresses during reperfusion or whether it occurs continuously throughout the course of I/R. A study by Fliss and Gattinger compared the timing and extent of apoptosis under both continuous ischemia and ischemia followed by reperfusion (Fliss H, et al., Circ Res 1996; 79: 949-956). The results suggest that ischemia alone can cause apoptosis, but reperfusion seem to accelerate the process.

A variety of methods have been devised to protect myocardium against injury during I/R. These include pretreatment with eicosanoid metabolites of arachidonic acid, a cocktail of glucose-insulin-potassium, caspase inhibitors that block apoptosis, G-actin sequestering peptide thymosin 4, nitric oxide (NO) precursors or NO donating agents, and S1P (Karliner J S, et al., J Mol Cell Cardiol 2001; 33(9): 1713-7; Bock-Marquette I, et al., Nature 2004; 432: 466-472; Spiecker M, et al., Arch Biochem Biophys 2005; 433: 413-420; Zhang H, et al., Apoptosis 2004; 9: 777-783; Yaoita H, et al., Circulation 1998; 97: 276-281; Yeh C, et al., J Thorac Cardiovasc Surg 2004; 128: 180-188; Kim J, et al., Free Radic Biol Med 2004; 37: 1943-1950). One of most effective means of minimizing I/R damage to the myocardium is ischemic preconditioning (IPC) in which a short sub-lethal period of I/R precedes the longer, more damaging treatment. SK activity is upregulated during IPC and is required for the cardioprotection imparted by IPC (Jin Z, et al., Circulation 2004; 110: 1980-1989; Lecour S, et al., J Mol Cell Cardiol 2002; 34(5): 509-18; Jin, Z, et al., Am J Physiol Heart Circ Physiol 2002; 282: H 1970-7).

Certain presently disclosed embodiments involve inhibiting the removal of S1P via SPL-mediated catalysis, through the use of inhibitors of SPL activity, to provide a powerful method for raising S1P levels and thereby artificially producing the beneficial effects of ischemic preconditioning on heart tissue prior to or during significant ischemic events.

The agents that inhibit SPL activity for use according to presently disclosed embodiments can also be used for the prevention and treatment of radiation and chemotherapy-induced cardiotoxicities. The cardiotoxicities associated with anthracyclines are mediated in large part by their ability to damage DNA, block DNA repair and generate reactive oxygen species, resulting in peroxidation of membrane lipids and mitochondrial DNA and subsequent activation of apoptotic pathways (Kalyanaraman B, et al., Mol Cell Biochem 2002; 234-235: 119-124; Feuerstein G Z and Young P R, Cardiovasc Res 2000; 45: 560-569; Arola O J, et al., Cancer Res 2000; 60: 1789-1792). This process appears to be p53-dependent, in that apoptosis and cardiac dysfunction after doxorubucin are absent in p53 knockout mice compared to severe effects in wild type littermate controls (Shizukuda Y, et al., Mol Cell Biochem 2005; 273: 25-32).

Among the signaling pathways activated by anthracyclines are sphingomyelin breakdown products including ceramide, which mediates cell death pathways involving components of the MAPK pathway (Laurent G and Jaffrezou JP, Blood 2001; 98: 913-924). Indeed, it has been shown that anthracycline-induced cardiac toxicity is dependent upon ceramide generation (Andrieu-Abadie N, et al., Faseb J 1999; 13: 1501-1510). Additional effects of chronic exposure to anthracyclines include changes in cardiac myosin heavy chain isoform composition and myofibril degeneration (Sussman M A, et al., Circ Res 1997; 80: 52-61).

The toxic cardiomyopathy induced by anthracyclines is exacerbated by combination therapy with trastuzumab (Herceptin), a humanized monoclonal antibody directed against the tyrosine kinase erbB2. While the etiology of trastuzumab's effects have not been fully elucidated, it significantly accentuates anthracycline-induced myofibrillar disarray (Sawyer D B, et al., Circulation 2002; 105: 1551-1554).

In contrast to anthracycline and trastuzumab mediated cardiotoxicity which is limited mainly to the myocardium, radiation therapy produces effects on myocardium, pericardium, valves and coronary vessels as well as the vasculature. Radiation is among the most potent stimuli of cardiomyocyte apoptosis, along with oxygen radicals, cytokines, the sphingolipid metabolite ceramide, and angiotensin II (Feuerstein G Z and Young P R, Cardiovasc Res 2000; 45: 560-569). Apoptosis of cardiac myocytes may contribute to progressive pump failure, arrhythmias and cardiac remodeling/fibrosis. In addition, endothelial cell apoptosis, which occurs through a p38-dependent pathway, is a significant factor contributing to organ damage from radiation (Kumar P, et al., J Biol Chem 2004; 279: 43352-43360; Brown W R, et al., Radiat Res 2005; 164: 662-668; Prise K M, et al., Lancet Oncol 2005; 6: 520-528).

As noted above, brief periods of cardiac ischemia significantly protect the myocardium against subsequent prolonged ischemic events. This phenomenon, known as ischemic preconditioning (IPC), leads to diminished infarct size, arrhythmias and left ventricular function after I/R (Williams R S and Benjamin I J, J Clin Invest 2000; 106: 813-818). Various mediators of IPC have been identified, including PKCε, p38, MAP kinases, PI3K and tyrosine kinases. Studies in which isolated cardiomyocyte cultures are exposed to conditions emulating I/R have demonstrated that IPC is a phenomenon intrinsic to myocardial cells (Diaz R J and Wilson G J, Cardiovasc Res 2006; 70(2): 286-96). Recently, it was demonstrated that S1P accumulation is an essential factor in mediating IPC, in that the enzyme responsible for S1P synthesis, sphingosine kinase (SK), is activated by IPC, leading to S1P elevation, and inhibition of SK abrogates the beneficial effects of IPC on cardiac hemodynamic function and myocardial tissue injury (Jin Z Q, et al., Circulation 2004; 110: 1980-1989; Karliner J S, et al., J Mol Cell Cardiol 2001; 33: 1713-1717; Jin Z, et al., Am J Physiol Heart Circ Physiol 2002; 282: H1970-1977).

The molecular effects of S1P signaling include interference with caspase activation, stimulation of Akt and MAP kinase signaling, and enhanced nitric oxide production (Saba J and Hla T, Circ Res 2004; 94: 724-734). These effects underlie the ability of S1P to protect mouse oocytes and other cells against doxorubicin, radiation and other toxic insults and may also be responsible for its cardioprotective role (Paris F, et al., Nature Medicine 2002; 8: 901-902; Awad A S, et al., Am J Physiol Renal Physiol 2006; Finigan J H, et al., J Biol Chem 2005; 280: 17286-17293; Perez G I, et al., Nat Med 1997; 3: 1228-1232).

Interestingly, IPC protection of heart function and tissue recovery was recently shown to extend to cardiomyocyte apoptosis induced by anthracyclines (Chicco A J, et al., J Appl Physiol 2006; 100: 519-527; Ascensao A, et al., Am J Physiol Heart Circ Physiol 2005; 289: H722-731). Further, inhibition of p38 was shown to induce adult cardiomyocyte reentry into the cell cycle in this normally nonreplicating cell (Engel F B, et al., Genes Dev 2005; 19: 1175-1187). It was recently demonstrated that SPL reduces S1P, elevates ceramide and mediates apoptosis via p53 and p38 (Oskouian B, et al., Proc Natl Acad Sci USA. 2006 Nov. 14; 103(46):17384-17389; Reiss U, et al., J Biol Chem 2004; 279: 1281-1290), and that SPL expression leads to constitutive activation of p38. Without wishing to be bound by theory, SPL inhibition may therefore afford cardioprotection by two mechanisms, first by providing “toxic preconditioning” through accumulation of S1P and thereby blocking apoptosis, and second by reducing p38 activation and thereby stimulating tissue regeneration.

The compositions and methods described herein, which relate to therapeutically beneficial inhibition of SPL, may be used for the prevention and treatment of cardiac injury due to coronary obstruction, percutaneous interventions, coronary artery bypass surgery, cardiopulmonary bypass, or non-cardiac surgery, repair of congenital heart disease with or without cardiopulmonary bypass, closure of septal defects by percutaneous means, percutaneous mitral valve repair or mitral valvulotomy, acute ischemia or hypoxia with or without reperfusion or reoxygenation, myocardial infarction of any cause, chronic congestive heart failure of any cause, myocarditis of any cause, cardiotoxicity from drugs such as doxorubicin, radiation, rastuzamab, and injury due to heart transplantation. Thus, the present invention provides compositions and methods for treating any cardiac failure resulting from cellular injury, from any apoptotic or necrotic process, including congestive heart failure from any number of infectious, toxic, genetic/inborn errors or other metabolic causes.

Although initially limited to balloon angioplasty and termed percutaneous transluminal coronary angioplasty (PTCA), percutaneous coronary interventions (PCIs) now includes other new techniques capable of relieving coronary narrowing. Accordingly, rotational atherectomy, directional atherectomy, extraction atherectomy, laser angioplasty, implantation of intracoronary stents and other catheter devices for treating coronary atherosclerosis are considered components of PCI.

In certain embodiments, the SPL inhibiting compositions described herein can be used to prevent or treat cardiac injury due to iron overload: The manifestations of iron overload are relatively uniform, irrespective of cause. Cardiac dysfunction is a primary cause of death in people with iron overload. Causes of iron overload include but are not limited to hereditary hemochromatosis and transfusion iron overload.

The SPL inhibiting agents described herein can be used for the prevention and treatment of injury due to stroke. Thus, the agents of the present invention are useful for the prevention and treatment of ischemia/reperfusion injury due to bleeding or embolus. Further, the agents of the present invention may be used for the prevention and treatment of acute ischemia or hypoxia with or without reperfusion or reoxygenation.

According to certain herein-disclosed embodiments, agents that inhibit SPL, such as those described herein, are also useful for the prevention and treatment of toxic dementias. In this regard, several signs of toxic dementia are memory impairment, deterioration of social and intellectual behavior, and attention deficits (Allen et al., 2001; Jacques, 1992; Headlee, 1948). Dementia is a general loss of cognitive abilities, including impairment of memory as well as one or more of the following: aphasia, apraxia, agnosia, or disturbed planning, organizing, and abstract thinking abilities. It does not include loss of intellectual functioning caused by clouding of consciousness (as in delirium), depression, or other functional mental disorder (pseudodementia).

Causes of dementia include a large number of conditions, some reversible and some progressive, that result in widespread cerebral damage or dysfunction. The most common cause is Alzheimer's disease; others include cerebrovascular disease, vascular dementia, central nervous system infection, brain trauma or tumors, vitamin deficiencies, anoxia, metabolic conditions, endocrine conditions, immune disorders, prion diseases, Wernicke-Korsakoff syndrome, normal-pressure hydrocephalus, Huntington's chorea, multiple sclerosis, and Parkinson's disease. SPL-inhibiting agents, such as those that are disclosed for use according to the several embodiments described herein, are useful for the prevention and treatment of neurotoxicity arising from any cause, including neuronal excitotoxicity, apoptosis and/or necrosis.

According to certain other embodiments provided herein, ischemia/reperfusion injury may be prevented or reduced in a subject by administering an agent that alters SPL activity, in a setting for treating or preventing transplantation related ischemia/reperfusion. Such grafts may, by way of non-limiting examples, be solid organ grafts (e.g., heart, kidney, etc.) or may alternatively be hematopoietic grafts, and graft rejection may be determined according to accepted criteria in the relevant medical arts, including classic host rejection of, e.g., allograft or xenograft tissue, and also including, e.g., graft-versus-host disease (GVHD) as may, for instance, accompany bone marrow transplantation following myeloablation in a therapeutic regimen for treating cancer. In certain embodiments, the present invention provides methods and compositions for inhibiting SPL to prevent intraoperative heart failure or to protect hearts used for transplantation.

The compositions and methods of the present invention are useful for the prevention and treatment of toxicity (including excitotoxicity), hypoxia, trauma, inflammatory, ischemic and other injuries to cardiac and/or CNS tissues such as those described herein in any subject known to be at risk for sustaining such an injury. As used herein, “subject” includes any mammal, including but not limited to humans, nonhuman primates, dogs, cats, mice, rats, guinea pigs, pigs, etc. As would be understood by the skilled artisan (e.g. a clinician), a subject at risk of sustaining a cardiac, stroke, or ischemia/reperfusion injury can be determined using known diagnostic criteria and procedures. Diagnosis of cardiac, stroke or other tissue injury contemplated herein in a subject having or suspected of being at risk for such an injury may be accomplished by any of a wide range of art-accepted methodologies, which may vary depending on a variety of factors including clinical presentation, degree of injury, the type of injury, family history and other factors. Diagnosis and determination of risk of injury is well within the ability of the skilled artisan and must be determined on a case by case basis.

Thus, the present invention provides methods for reducing or preventing cardiac injury in a subject known to have, or to be at risk for sustaining, cardiac injury, comprising administering to the subject an agent that inhibits sphingosine-1-phosphate lyase (SPL) activity and thereby reducing or preventing cardiac injury in the subject. In a further aspect, the present invention provides methods for reducing or preventing stroke injury in a subject known to have, or to be at risk for sustaining, stroke injury, comprising administering to the subject an agent that inhibits sphingosine-1-phosphate lyase (SPL) activity and thereby reducing or preventing stroke injury in the subject. In another aspect, the present invention provides methods for preventing or reducing tissue injury due to organ transplantation in a subject, comprising administering to the subject an agent that inhibits sphingosine-1-phosphate lyase (SPL) activity and thereby preventing or reducing tissue injury due to organ transplantation. Generally, the present invention also provides methods for reducing or preventing ischemia/reperfusion injury in a tissue in a mammal comprising, administering to said mammal an agent that inhibits SPL activity.

For in vivo use, an inhibiting agent as described herein is generally incorporated into a pharmaceutical composition prior to administration. A pharmaceutical composition comprises one or more inhibiting agents in combination with a physiologically acceptable carrier. To prepare a pharmaceutical composition, an effective amount of one or more inhibiting agents is mixed with any pharmaceutical carrier(s) known to those skilled in the art to be suitable for the particular mode of administration. A pharmaceutical carrier may be liquid, semi-liquid or solid. Solutions or suspensions used for parenteral, intradermal, subcutaneous or topical application may include, for example, a sterile diluent (such as water), saline solution, fixed oil, polyethylene glycol, glycerine, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously, suitable carriers include physiological saline or phosphate buffered saline (PBS), and solutions containing thickening and solubilizing agents, such as glucose, polyethylene glycol, polypropylene glycol and mixtures thereof. In addition, other pharmaceutically active ingredients (including other anti-cancer agents) and/or suitable excipients such as salts, buffers and stabilizers may, but need not, be present within the composition.

An inhibiting agent of the present invention may be prepared with carriers that protect it against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

Administration may be achieved by a variety of different routes, including oral, parenteral, nasal, intravenous, intradermal, subcutaneous or topical. Preferred modes of administration depend upon the nature of the condition to be treated or prevented. An amount that, following administration, inhibits, prevents or reduces cardiac, stroke, or ischemia/reperfusion injury is considered effective. The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

As an alternative to direct administration of an inhibiting agent, a polynucleotide encoding an inhibiting agent may be administered. Such a polynucleotide may be present in a pharmaceutical composition within any of a variety of delivery systems known to those of ordinary skill in the art, including nucleic acid, bacterial and viral expression systems, and colloidal dispersion systems such as liposomes. Appropriate nucleic acid expression systems contain the necessary DNA sequences for expression in the patient (such as a suitable promoter and terminating signal, as described above). The DNA may also be “naked,” as described, for example, in Ulmer et al., Science 1993; 259: 1745-49.

Various viral vectors that can be used to introduce a nucleic acid sequence into the targeted patient's cells include, but are not limited to, vaccinia or other pox virus, herpes virus, retrovirus, or adenovirus. Techniques for incorporating DNA into such vectors are well known to those of ordinary skill in the art. Another delivery system for polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preparation and use of liposomes is well known to those of ordinary skill in the art.

Within certain aspects of the present invention, one or more inhibiting agents may be used to modulate SPL expression and/or activity, including, for example, in vitro, in a cell that is ex vivo, in vitro or in vivo, or in a mammal, including humans and non-human mammals. In vitro, an SPL polypeptide may be contacted with an inhibiting agent that increases or decreases SPL activity (e.g., certain antibodies). For use within a cell or a mammal, such modulation may be achieved by contacting a target cell with an effective amount of an inhibiting agent, as described herein. Administration to a mammal may generally be achieved as described above.

An inhibiting agent may be prepared as described herein and may in certain embodiments be used in combination with other therapies including, but not limited to, beta-blockers (e.g., Acebutolol (Sectral); Atenolol (Tenormin); Betaxolol (Kerlone); Bisoprolol (Zebeta); Carvedilol (Coreg); Labetalol (Normodyne, Trandate); Metoprolol succinate (long acting Toprol XL); Metoprolol tartrate (Lopressor); Nadolol (Corgard); Penbutolol (Levatol); Pindolol (Visken); Propranolol (Inderal); Propranolol long-acting (Betachron, Inderal-LA, Innopran XL); Timolol (Blocadren)) and/or antioxidants (such as, but not limited to Alpha Lipoic Acid, Beta-carotene, Ubiquinone, Cucurmin, Cysteine, Glutathione, Oligomeric Proanthocyanidins, Pychnogenol, Selenium, Vitamin A, C, E, Zinc), immunosuppressants (such as, but not limited to, azathioprine, basiliximab, daclizumab, sirolimus, tacrolimus, muromonab-CD3, cyclophosphamide, mycophenolate, cyclosporine, methotrexate and mercaptopurine), anticoagulants (e.g., heparin; warfarin), antiplatelets (e.g., aspirin; clopidogrel, an antiplatelet/platelet aggregation inhibitor drug that is used to help prevent second strokes; dipyridamole; ticlopidine), etc.

The following Examples are offered by way of illustration and not by way of limitation.

### EXAMPLES

### Example 1

### Inhibition of SPL by THI Prevents Cardiac Ischemic Injury

This example shows cardioprotective effects conferred by administration of an agent that inhibits SPL activity. As a brief background, it was shown previously that SPL expression enhances apoptotic responses to serum deprivation by elevating ceramide and reducing S1P (Reiss U, et al., J Biol Chem 2004; 279: 1281-1290). Also, it was recently found that overexpression of SPL is associated with constitutive activation of p38 MAP kinase and that activation of both p38 and p53 are required for SPL induced apoptosis, as shown by attenuation of the effect by the p53 inhibitor pifithrin α and the p38 inhibitor SB203580 (Oskouian B, et al., Proc Natl Acad Sci USA. 2006 Nov. 14; 103(46):17384-17389). Since p38 inhibition promotes cardiomyocyte survival and reentry into cell cycle, SPL inhibition may prevent p38 activation and promote cell survival and tissue regeneration in the face of cardiotoxic injury to the myocardium.

The small molecule agent tetrahydroxy-butylimidazole (THI) inhibits SPL and raises tissue S1P levels after oral administration in mice (Schwab S, et al. Science 2005; 309: 1735-1739). To determine whether SPL inhibition might afford protection against ischemia/reperfusion (I/R) injury, left ventricular (LV) function and infarct size were compared in hearts isolated from control and THI-treated mice after experimentally induced I/R injury. C57BL6 male mice were given THI in the water supply for 3 days. This led to SPL inhibition and elevated S1P levels in most tissues, including the heart, which increased from 0.17 to 0.33 μmol/μg phosphatidylcholine. After 50 min no-flow ischemia, followed by 40 min reperfusion, LVEDP (LV end-diastolic pressure, an indicator of heart function and residual fluid volume at the end of diastole) was 52 mmHg in the control heart, whereas it was 34 mmHg in the THI-treated heart. LVDP (LV developed pressure, a measure of cardiac function) was only 22 mmHg in control mouse, whereas in the THI-treated mouse, it was 52 mmHg. Importantly, the size of infarction (determined by tissue uptake of tetrazolium red, TTC) was 35% in the control heart and 17% in the THI-treated heart. Two other treated vs. control hearts showed similar results (Table 1). These results suggest SPL blockade may afford cardiac protection against I/R injury.

TABLE 1
LCDP (Left Ventricular Developed Pressure)

| Condition | Animal | Baseline | Reperf. 40′ | % Recovery |
|---|---|---|---|---|
| Control | 1 | 94 | 22 | 23.4 |
| | 2 | 126 | 16 | 12.7 |
| | 3 | 113 | 34 | 30.1 |
| | Ave. | 111.0 | 24.0 | 22.1 |
| | SD | 16.1 | 9.2 | 8.8 |
| | SEM | 9.3 | 5.3 | 5.1 |
| | p | <.02 vs Reper | <.03 vs THI | <.03 vs THI |
| THI | 1 | 122 | 52 | 42.6 |
| | 2 | 137 | 88 | 64.2 |
| | 3 | 140 | 99 | 70.7 |
| | Ave. | 133.0 | 79.7 | 59.2 |
| | SD | 9.6 | 24.6 | 14.7 |
| | SEM | 5.6 | 14.2 | 8.5 |
| | p | <.03 vs Reperf | | |

### Example 2

### SPL-Promoted Cell Death in Response to Doxorubicin In Vitro

This example shows enhancement of the cytotoxic effect of the chemotherapeutic agent doxorubicin when cells overexpress SPL. HEK294 cells expressing either pcDNA3 vector (PC) or a human SPL cDNA construct (hSPL) were incubated for 24 h (black bars) or 48 h (white bars) with doxorubicin, harvested and viability was determined by MTT assay. (Mosmann, J Immunol Methods, 1983, 65(1-2): 55-63).

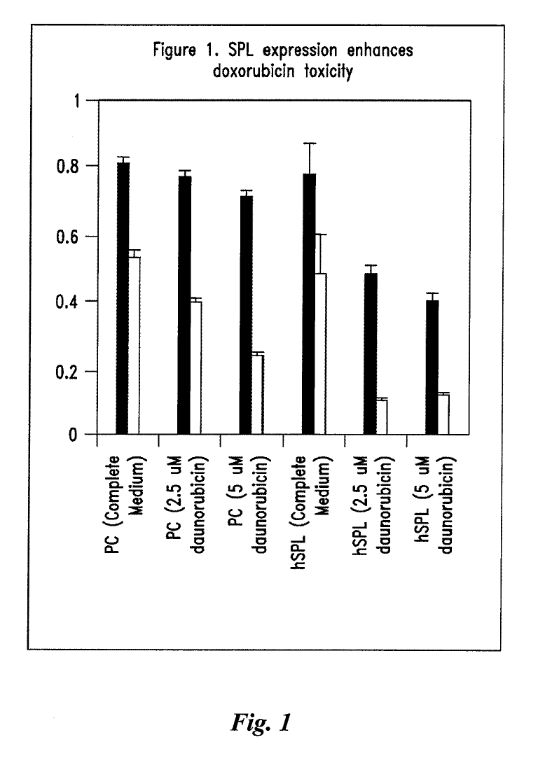

As shown in FIG. 1, cell survival in the presence of doxorubicin was significantly reduced in cells overexpressing SPL. These results suggested that inhibition of SPL may provide a cytoprotective strategy against doxorubicin cytotoxicity.

### Example 3

### Molecular Modulation of SPL Expression Using Adenoviral Human-SPL and Species-Specific SPL-siRNA

The human SPL gene was cloned in KpnI/XhoI sites of pAdTrack-CMV and used for virion production. This vector expressed a GFP control from a separate promoter, allowing efficiency of infection to be assessed. Adenoviruses were propagated in Ad-293 cells (Stratagene, La Jolla, Calif.), purified using VivaPure AdenoPack 100 from VivaScience (Hanover-Germany) and used to infect cardiomyocytes 24 h after isolation at a MOI of 100. Infection of cells with Ad-SPL using this system was >90%. SPL protein expression was markedly enhanced, as determined by immunoblotting extracts from Ad-SPL versus Ad-GFP infected cells. SPL activity in control cardiomyocytes contained 13 pmol/mg/min SPL activity, whereas cells containing the SPL construct contained 280 pmol/mg/min activity.

Similar experiments are used to achieve high SPL expression in HUVECs, which have low baseline SPL activity. In order to achieve knockdown of mouse SPL the pSilencer adeno 1.0-CMV system developed by Ambion (Austin, Tex.) is used. Briefly, a double stranded oligonucleotide encoding the following 21 bases is cloned: TATGAGCCCTGGCAGCTCATT (SEQ ID NO:22) followed by a 6 base loop followed by the complementary sequence into shuttle vector adeno 1.0-CMV. This vector along with an adenoviral backbone vector is linearized and transfected into HEK293 cells to allow for recombination and the generation of the viral particles. The chosen sequence corresponds to mouse SPL sequences starting at nucleotide 107 from the start codon and has been reported to effectively knock down SPL expression (Pettus B J, et al., Faseb J 2003; 17: 1411-1421). For knockdown of human SPL in HUVECs, oligomers were designed using Dharmacon siDesign Center:

Top

(SEQ ID NO:23)

5′-TCGAGGCATACTGATGGCCTGCAATTCAAGAGATTGCAGGCCATCAG
TATGCTTA-3′
Bottom

(SEQ ID NO:24)

5′-CTAGTAAGCATACTGATGGCCTGCAATCTCTTGAATTGCAGGCCATC
AGTATGCC-3′

The target starts at position 848 in human SPL cDNA. Adenoviral infection of HUVECs is as described (Riccioni T, et al., Gene Ther 1998; 5: 747-754).

### Example 4

### In Vivo and In Vitro Models of SPL Expression

In Vivo Models:

A SPL knockout mouse model was generated from ES cells with a targeted SPL deletion (Chen W, et al., Nature Genetics 2004; 36: 304-312). Homozygous knockout pups survived 1-2 weeks postnatally and succumbed for unknown reasons. SPL protein was expressed in all tissues of wild type animals that were examined, whereas knockout mouse tissues had no detectable SPL expression, in good correlation with detectable levels of SPL enzyme activity. S1P levels were vastly elevated in SPL knockout tissues (knockout hearts had 2088 ng/g wet weight S1P compared to 4.8 ng/g in wild type and 14 ng/g in heterozygotes).

SPL expression was examined in various tissues of wild type (SPL^{+/+}) and SPL knockout (SPL^{-/-}) pups using western immunoblotting with an antibody specific for SPL. SPL was expressed in wild type heart tissue, and knockout mouse tissues, including heart, had virtually no detectable SPL expression. These findings correlated well with detectable SPL enzyme activity levels the tissues examined. As shown in Table 2 below, S1P levels were vastly elevated in SPL knockout tissues.

TABLE 2
Tissue S1P Levels in SPL^{+/+}, SPL^{+/-} and SPL^{-/-} Mice

| Tissue | SPL^{+/+} | SPL^{+/-} | SPL^{-/-} |
|---|---|---|---|
| Intestine | 4.93 | 3.23 | 1338.01 |
| Stomach | 85.30 | 55.36 | 545.16 |
| Heart | 4.82 | 14.11 | 2088.87 |
| Lung | 33.29 | 35.47 | 2889.36 |
| Spleen | 19.43 | 27.30 | 2267.11 |
| Muscle | 4.11 | 5.02 | 320.82 |
| Brain | 43.55 | 26.21 | 74.25 |
| Liver | 3.73 | 2.93 | 1137.70 |
| Thymus | 13.01 | 4.26 | 1172.50 |
| Kidney | 25.91 | 8.33 | 2416.47 |
| Colon | 10.41 | 15.23 | 586.43 |

SPL expression in the mammalian heart was therefore demonstrated by these results. The SPL knockout mouse is therefore contemplated for the preparation of primary cardiomyocytes, providing a model in which to examine the role of SPL in cardiac preconditioning, and hypoxic injury.

Other mouse models are also used for the studies described herein. For example, Adult C57BL/6 male mice and mouse pups less than 24 h of age are purchased from Charles River Laboratories. Mice heterozygous for the SPL null allele are as described by Soriano (Nature Genetics 2007 January; 39(1):52-60). These mice are a mixed C57BL/6 and 129SvEvTac line, requiring the use of littermate controls for all experiments. Genotyping is performed on genomic DNA isolated from 0.5 cm mouse tail. Tissue is incubated for 4-6 h in buffer (50 mM Tris-Hcl pH 8.0, 25 mM EDTA, 100 mM NaCl, 1% SDS) plus proteinase K, followed by phenol and chloroform extractions, ethanol precipitation, 70% ethanol wash, drying and resuspension in TE buffer. For SPL genotyping, primers Sgpl-1F (5′ CGC TCA GM GGC TCT GAG TCA TGG 3′) (SEQ ID NO:25) and Sgpl-1R (5′ CCA AGT GTA CCT GCT MG TTC CAG 3′) (SEQ ID NO:26) for wild type or Sgpl1-1F and SARev (5′ CAT CAA GGA AAC CCT GGA CTA CTG 3′) (SEQ ID NO:27) for knockout alleles are used in a reaction with Taq polymerase (95° C.—5′ denaturation, followed by 95° C. 1′, 55° C. 1′, and 72° C. 3′ for 40 cycles). Presence of both wild type and mutant alleles indicates heterozygosity, whereas single wild type or mutant products indicate homozygosity for wild type or mutant genotype respectively. Mice are maintained using standard mouse husbandry.

In Vitro Models:

Cardiomyocytes with low, moderate or high S1P levels are isolated from pups of different genotypes and are used as an in vitro system to test the impact of S1P levels and SPL expression on protection from cardiac injury due to I/R, cardiotoxic therapeutic interventions such as those used in cancer treatment, hypoxia, etc.

Further experiments are performed to determine viability and apoptotic responses to stressful in vitro growth conditions associated with I/R. In particular, viability and apoptotic responses to stressful in vitro growth conditions associated with I/R are compared in primary murine cardiomyocytes and fibroblasts exhibiting different levels of SPL expression and activity. Generally, viable cardiac myocytes and fibroblasts can be isolated from mice of different ages, from day 1 of life to several months of age. This makes it possible to compare the survival under stressful conditions of cells with different levels of SPL activity by virtue of: 1) molecular modulation of SPL expression in wild type neonatal cardiomyocytes and fibroblasts using adenoviral and lentiviral vectors, 2) isolation of cardiomyocytes and fibroblasts from 1-2 week old SPL +/+, −/− and +/− littermates, and 3) isolation of cardiomyocytes and fibroblasts from young, wild type mice treated with THI or vehicle. To avoid differences resulting from age (neonatal vs. adult myocytes), all experiments are performed on age-matched controls.

Primary myocytes are isolated using a preplating technique, which allows fibroblasts to adhere to the plates, thereby enriching myocytes in the passaged culture (see e.g., Karliner J, et al. J Mol Cell Cardiol 2000; 32:1779-1786; and Xian M, et al. J Mol Cell Cardiol 1999; 31:2155-2165). Briefly, primary adult and neonatal cardiac myocyte cultures are prepared by enzymatic digestion of ventricular tissue from buffer-perfused mouse hearts essentially as described (Karliner J S, et al. J Mol Cell Cardiol 2001; 33(9):1713-7; Karliner et al. 2000 Supra). This protocol yields approximately 80% rod-shaped myocytes at a plating density of 50 cells/mm². Neonatal cells are studied from 1-2 days up to 3-4 days after initial culture. Adult cells are studied the day after isolation and culture, as described (Tao R, et al., Cardiovasc Res 2007; 74(1):56-63).

Primary neonatal cardiomyocytes are also isolated from genetically altered mouse lines including the SPL knockout line (129SvEv background), the p53 knockout line (C57BL/6J background) and the p38 knockout line (C57BL/6J background), along with wild type and in some cases heterozygous controls from the same genetic backgrounds. In cases where homozygotes are viable and breed well, several litters are combined from each individual genotype to yield sufficient cardiomyocytes for molecular modulation by SPL-overexpressing, SPL-knockdown and control adenoviral plasmids. In the case of SPL knockout models, pups born to heterozygote parents will be sacrificed, genotyped and harvested individually to keep genotypes separate. All pups are euthanized on day of life 1 by decapitation and heart tissues are harvested. These studies are complemented by TUNEL and annexin staining of cardiac tissues in situ.

Wild type, heterozygous and homozygous p53 mutant mice (Trp53tm1Tyj strain in the C57BL/6J background) are available from Jackson Laboratories. The cardiomyocyte-specific p38α knockout line was generated by crossing floxed-p38α knockout mice with the MLC2a/Cre transgenic line, which expresses Cre in a cardiac-specific manner (Engel F B, et al., Genes Dev 2005; 19: 1175-1187). Inhibition of p53 with pifithrin α (Sigma) and p38 with SB203580 (Calbiochem) is performed as described.

SPL expression is determined using quantitative RT-PCR and immunoblotting with a polyclonal antiserum against a C-terminal peptide of the murine SPL protein which is both sensitive and specific. A biochemical assay is used to measure SPL activity. This assay relies on following the formation of radioactive aldehyde product, which is separated from substrate by thin layer chromatography and quantitated by scintillation counting. S1P quantitation is performed using routine HPLC and MS techniques. Results are normalized to phosphatidylcholine as an indicator of total lipid in each sample. SPL expression is modulated using plasmid and adenoviral expression systems (Ad-SPL or Ad-SPL siRNA) as described elsewhere herein (see Example 3 and Example 11).

Effects of altered SPL activity on cell viability and apoptosis is determined at baseline and in response to serum deprivation, hypoxia, oxidative stress (using H₂O₂, menadione and other oxidants) and glucose deprivation. Survival time courses are established in wild type cells first, and subsequent experiments are timed to identify deviations from the LD₅₀ associated with altered SPL expression. For proliferating cells, viability is determined by MTT assay, which relies on reduction of 3-(4,5-dimethylthiazol-2-yl)-2,5-diphenyl tetrazolium bromide (MTT reagent) by viable cells. The optical density of reduced MTT is followed spectrophotometrically. For adult and neonatal cardiac myocytes, which do not proliferate, viability is determined by trypan blue exclusion. Apoptosis is measured by activation of caspase-3 and immunoblotting of cytosolic extracts to follow cleavage of the caspase-3 substrate PARP. Viability (of proliferating cell types) and apoptosis are performed as described (Oskouian B, et al. 2006 Proc. Natl. Acad. Sci. USA November 14; 103(46):17384-9). For cardiac myocytes, viability is quantified by treating cells with trypan blue diluted to a final concentration of 0.04% (w/v) and counting stained (non-viable) and unstained (viable) cells, as described (Tao R, et al., Cardiovasc Res 2007; 74(1):56-63). Alternatively, the Eukolight Viability/Cytotoxicity assay (Molecular Probes, Eugene, Oreg.) may be employed. as described (Gray M O, et al., J Biol Chem 1997; 272(49):30945-51).

### Example 5

### Determining SPL Expression, Activity, and S1P Levels

SPL activity is measured using any of a variety of assays, such as those described in U.S. application Ser. Nos. 08/939,309, 10/053,510, and 10/348,052.

In certain embodiments, the SPL activity of an SPL polypeptide or variant thereof is assessed using an in vitro assay that detects the degradation of labeled substrate (i.e., sphingosine-1-phosphate, or a derivative thereof). Within such assays, pyridoxal 5′-phosphate is a requirement for SPL activity. In addition, the reaction generally proceeds optimally at pH 7.4-7.6 and requires chelators due to sensitivity toward heavy metal ions. The substrate should be a D-erythro isomer, but in derivatives of sphingosine-1-phosphate the type and chain length of sphingoid base may vary. In general, an assay as described by Van Veldhoven and Mannaerts, J Biol Chem 1991; 266: 2502-07, may be employed. Briefly, a solution (e.g., a cellular extract) containing the polypeptide may be incubated with 40 μM substrate at 37° C. for 1 hour in the presence of, for example, 50 mM sucrose, 100 mM K-phosphate buffer pH 7.4, 25 mM NaF, 0.1% (w/v) Triton X-100, 0.5 mM EDTA, 2 mM DTT, 0.25 mM pyridoxal phosphate. Reactions may then be terminated and analyzed by thin-layer chromatography to detect the formation of labeled fatty aldehydes and further metabolites. In general, a polypeptide has SPL activity if, within such an assay: (1) the presence of 2-50 μg polypeptide (or 0.1-10 mg/mL) results in a statistically significant increase in the level of substrate degradation, preferably a two-fold increase, relative to the level observed in the absence of polypeptide; and (2) the increase in the level of substrate degradation is pyridoxal 5′-phosphate dependent.

Within certain embodiments, an in vitro assay for SPL activity may be performed using cellular extracts prepared from cells that express the polypeptide of interest. Preferably, in the absence of a gene encoding an SPL polypeptide, such cells do not produce a significant amount of endogenous SPL (i.e., a cellular extract should not contain a detectable increase in the level of SPL, as compared to buffer alone without extract). It has been found, within the context of the present invention, that yeast cells containing deletion of the SPL gene (BST1) are suitable for use in evaluating the SPL activity of a polypeptide. bst1Δ cells can be generated from S. cerevisiae using standard techniques, such as PCR, as described herein. A polypeptide to be tested for SPL activity may then be expressed in bst1Δ cells, and the level of SPL activity in an extract containing the polypeptide may be compared to that of an extract prepared from cells that do not express the polypeptide. For such a test, a polypeptide is preferably expressed on a high-copy yeast vector (such as pYES2, which is available from Invitrogen) yielding more than 20 copies of the gene per cell. In general, a polypeptide has SPL activity if, when expressed using such a vector in a bst1Δ cell, a cellular extract results in a two-fold increase in substrate degradation over the level observed for an extract prepared from cells not expressing the polypeptide.

A further test for SPL activity may be based upon functional complementation in the bst1Δ strain of Saccharomyces cerevisiae (Saba et al., 1997 J. Biol. Chem. 272:26087). It was been found, within the context of the presently disclosed embodiments, that bst1Δ cells were highly sensitive to D-erythro-sphingosine. In particular, concentrations as low as 10 μM sphingosine completely inhibited the growth of bst1Δ cells. Such a level of sphingosine had no effect on the growth of wildtype cells. A polypeptide having SPL activity as described above significantly diminished (i.e., by at least two fold) the sphingosine sensitivity when expressed on a high-copy yeast vector yielding more than 20 copies of the gene per cell.

SPL expression and activity is also measured using Western immunoblot analysis and immunohistochemistry using anti-SPL antibodies and standard techniques known in the art as described, for example, in Bandhuvula P, et al. J Biol Chem 2005; 280:33697-33700.

In certain embodiments, SPL activity is measured in a standard assay utilizing a radioactive S1P substrate (American Radiolabeled Chemicals, Inc.). Whole cell extracts containing SPL activity are incubated with substrate for 1 h at 37° C., followed by extraction of product, separation by thin layer chromatography and scintillation counting (Bandhuvula P, et al., 2005 Supra). Sphingolipid measurements are performed by isolating lipids, derivatizing them with ortho-phthalaldehyde, separation on HPLC and detection by fluorescence detection as described by Jiang Q, et al., Proc Natl Acad Sci USA 2004; 101: 17825-17830. Western analysis is performed using polyclonal anti-mouse SPL antisera.

SPL activity can also be measured using a fluorescence-based assay with NBD-S1P as a substrate.

### Example 6

### In Vitro Model for Determining Cytotoxicity of Cytotoxic Therapy

This example describes demonstration in a cell-based in vitro system of the efficacy of SPL inhibitors as described herein in reducing or preventing radiation- and/or chemotherapy-induced cytotoxicity.

Doxorubicin at 0.1 or 0.5 μmol/L is administered alone or in combination with anti-rodent erbB2 receptor antibody (clone B10, AB9, NeoMarkers) or the erbB2 ligand neuregulin-1β at 1 μg/ml to cell cultures for 48 h, as previously described (Sawyer D B, et al., Circulation 2002; 105: 1551-1554). Both agents activate rodent erbB2 and exacerbate anthracycline toxicity in rat cardiomyocyte cultures. Dose and time course studies are performed to establish toxicity in cultured wild type myocytes using radiation, or the single or combined chemotherapeutic agents. The in vitro exposures are completed with 320 kVp Xrays at a dose-rate of 1.3 Gy/min after NIST-based dosimetry to confirm field uniformity. Experiment are completed in triplicate. Once toxicity curves are established for wild type cells, administration of therapy to cells containing high or low SPL expression and activity by virtue of modulation using adenoviral expression systems or genetic inheritance (SPL knockout myocytes) is performed. Resulting apoptosis and cell death are determined by MTT assay, caspase-3 activity assays in whole cell extracts, PARP cleavage and annexin binding and quantitation by flow cytometry as described (Reiss U, et al., J Biol Chem 2004; 279: 1281-1290; Jiang Q, et al., Proc Natl Acad Sci USA 2004; 101: 17825-17830).

### Example 7

### In Vivo Model for Determining Cytotoxicity of Cytotoxic Therapy

This example describes in vivo protection against cardiotoxic effects of chemotherapy and radiation therapy using an SPL inhibitor. Oral administration to mice of the food additive THI for three days resulted in profound inhibition of SPL activity in most organs and concomitant increases in the tissue levels of S1P. S1P itself has been shown to protect oocytes and other cell types against the toxicity of radiation, chemotherapy and other stressful conditions. Therefore, administration of THI to cancer patients prior to receiving cardiotoxic therapies may protect the myocardium from life-threatening side-effects.

THI with 10% glucose (to avoid unpalatable taste) is administered to adult C57BL/6J mice as described in Example 1 (see Schwab et al., Science 2005; 309: 1735-1739). SPL inhibition and S1P measurements are assessed as described herein using assays known in the art.

Doxorubicin (NovaPlus, Bedford Ohio) is administered by injection at 15 mg/kg via tail vein, as described (Bernstein D, et al., Am J Physiol Heart Circ Physiol 2005; 289: H2441-2449). Mice are euthanized and cardiac tissues evaluated 24 hours after treatment. Pathological and biochemical analysis of cardiac tissues from dox-treated mice is performed. Dosage and schedule are titrated as needed to ensure survival of mice for the experimental period while obtaining significant pathology. Criteria for determining doxorubicin-induced pathology include: (a) reduction in left-ventrical function as determined by echocardiogram; (b) death as an endpoint of cardiotoxicity; (c) pathology as determined by histological examination; and (d) diastolic/systolic pressure as determined by the Langendorff “hanging heart” method.

Studies are then repeated using the so-refined treatment regimen in two separate groups, 1) a control group pretreated with glucose alone, and 2) an experimental group pretreated with THI/glucose for three days, followed by treatment of both groups with doxorubicin. Similar studies are performed with mice irradiated in single, acute partial body exposures of the mid-chest using lead shielding and a 320 kVp x-ray unit. NIST-based dosimetry is performed prior to each experiment to confirm uniform radiation fields and the dose rate of 1.3 Gy/min. The mice are restrained without anesthesia during the exposure. After a first dose-range study, experiments are completed at a selected single sublethal dose with confirmed cardiac pathology.

### Example 8

### Ex Vivo Model System for Measurement of Cardiac Injury

### Langendorff Isolated Perfused Heart Preparations and Left Ventricular Performance

This example describes use of an isolated perfused heart model system for assessing cardioprotective effects of SPL inhibitors. Materials and methods for preparing isolated and perfused murine hearts have been previously described (Jin Z Q, et al., Circulation 2004; 110: 1980-1989; Jin Z, et al., Am J Physiol Heart Circ Physiol 2002; 282: H1970-1977). Briefly, mice are heparinized (500 U/kg ip) and anesthetized with pentobarbital sodium (60 mg/kg ip). Hearts are rapidly excised, washed in ice-cold arresting solution (120 mmol/l NaCl, 30 mmol/l KCl), and cannulated via the aorta on a 20-gauge stainless steel blunt needle. Hearts are perfused at 90 mmHg on a modified Langendorff apparatus using Krebs-Henseleit solution containing 118 mM NaCl, 4.7 mM KCl, 2.5 mM CaCl₂, 1.2 mM MgSO₄, 1.2 mM KH₂PO₄, 24 mM NaHCO₃, 5.5 mM glucose, 5.0 mM Na pyruvate, 0.5 mM EDTA, and bubbled with 95% O₂-5% CO₂ at 37° C.

Platinum electrodes connected to a stimulus generator are used to pace hearts at 360 beats/min. Left ventricular developed pressure (LVDP)=LV systolic pressure−LV end diastolic pressure and is measured using a 1.4-Fr micromanometer passed into a polyvinylchloride film balloon filled with water to set the LVEDP at <10 mmHg. The balloon is inserted through the left atrium into the left ventricle, and pressures are recorded continuously on a chart recorder. Coronary flow is measured by collecting effluent from the coronary sinus. Noninvasive measurement of LV size and function are performed using standard echocardiographic methods in unanesthetized mice. LV dimensions, fractional shortening and ejection fraction are calculated before and at the end of a treatment period, before sacrifice in all animals.

In vivo hemodynamic function, animal survival endpoints, the Langendorff isolated heart functional assays and thorough pathological analysis of cardiac tissues are employed to address cardiac tissue injury, such as that induced by vascular phenomena including leukocyte plugging, compression from interstitial edema and contracture. Experimentally induced cardiotoxicity by, e.g., one or more chemotherapeutic agents (e.g., doxorubicin) and the cardioprotective effects of SPL inhibitors are assessed.

### Example 9

### SPL Expression in Response to Hypoxia

This example describes experiments showing that hypoxia induces SPL gene transcription and thus suggesting that SPL inhibition may protect cells from tissue damage arising due to sequelae of SPL upregulation. To dissect the molecular mechanisms regulating SPL expression a luciferase reporter system was established as described (Oskouian B, et al., J Biol Chem 2005; 280(18): 18403-10). SPL expression was then examined by performing transient transfection of the reporter constructs in a variety of cell lines with simultaneous co-transfection with RL-SV40, a plasmid which constitutively expresses a sea pansy luciferase gene allowing normalization of the transfection efficiency among different experiments. Transient transfections of pGL3-Kpn1 (i.e., 1.8 kb promoter fragment) into Hek293 and HeLa cells, followed by subjecting the transfected cells to hypoxia and measuring luciferase activity showed that hypoxia in either cell line upregulates the SPL promoter. The same phenomenon was observed in MCF-7, MBA-MD231 and HepG2 cells. It is possible that multicopy regulatory elements introduced into the cell by transient transfections might affect reporter activity, (for example, by titration of trans-activating factors), or that the regulation of a gene on an extrachromosomal plasmid might differ from one residing on the chromosome (due to chromatin structure affecting its availability to trans-activators). To address these issues, Hek293 cell lines were constructed that have chromosomally integrated copies of the reporter plasmids. When these cells were subjected to hypoxic conditions, again a higher luciferase activity was obtained compared to cells grown in normal oxygen, suggesting that the chromosomal copy of the SPL promoter was also upregulated by hypoxia. Also, overexpression of SPL in bovine aortic endothelial cells made them more sensitive to hypoxic conditions (<1% O₂), and an increase in apoptosis was observed as determined by an increase in PARP. Additional studies have established that both SPL reporters and endogenous SPL gene expression are upregulated by DNA damage. Thus, SPL expression is induced in response to cellular stress.

### Example 10

### SPL Expression in Skeletal Muscle, Heart and Other Cell Types

This example shows SPL expression in various cell types.

SPL gene expression has been shown to increase markedly in regenerating muring skeletal muscles after injury (see Zhao P, et al. J Biol Chem 2002; 277:30091-30101). Cardiac and skeletal muscles have overlapping but distinct biological functions and expression patterns. Therefore, to address whether SPL expression and activity might be relevant in cardiac physiology and the pathophysiology of I/R, Northern analysis and immunoblotting was performed to determine gene and protein expression in murine heart. These experiments confirmed SPL expression in murine heart (see Zhou J and Saba J. Biochem Biophys Res Commun 1998; 242(3):502-507. SPL expression was also observed in 3T3 fibroblasts and primary cardiac myocytes.

SPL activities of 2-5 μmol/mg/min were also observed in total murine cardiac tissue and 13±2 μmol/mg/min in murine cardiac myocytes.

Thus, SPL is expressed in cardiac tissues and cells relevant to cardiac I/R injury.

### Example 11

### SPL Alters Cellular Response to Hypoxia, Radiation and Daunorubicin

In further experiments, an adenoviral expression system was used to overexpress human SPL in bovine aortic endothelial cells (BAECs) and assess the effect of SPL on cell survival under hypoxic conditions. Since available poly ADP ribose polymerase (PARP) antibodies do not cross-react with bovine PARP, apoptosis was measured via the generation of PAR, the product of PARP, itself a substrate of caspase-3. In particular, BAECs expressing SPL or GFP were grown under normoxic (N) or hypoxic (H) conditions for 24 hours, and apoptosis was determined by abundance of poly(ADP-ribose) (PAR), the product of PARP in whole cell extracts. The results showed that overexpression of SPL in BAECs markedly increased apoptosis in response to hypoxia.

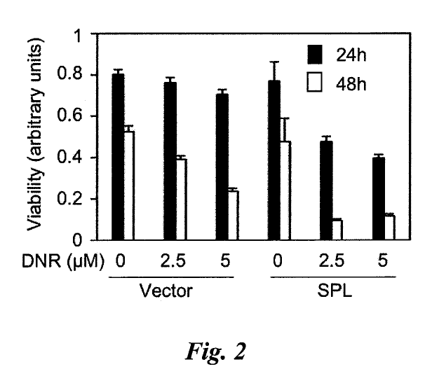

In an additional experiment, HEK293 cells expressing human SPL or vector control were treated with daunorubicin (DNR), and viability was determined by MTT assay. This assay relies on reduction of 3-(4,5-dimethylthiazol-2-yl)-2,5-diphenyl tetrazolium bromide (MTT reagent) by viable cells. HEK293 kidney cells stably overexpressing human SPL (hSPL) were more sensitive to the cardiotoxic agent daunorubicin than cells containing a vector control (FIG. 2).

SPL expression increases apoptosis in cells in response to hypoxia and increases cell sensitivity to cardiotoxic effects of daunorubicin. Thus, inhibiting SPL expression may have therapeutic benefits in a variety of cardiac injury and treatment settings.

### Example 12

### SPL is Rapidly Activated by Ischemia/Reperfusion Injury In Vivo

While some untoward effects of I/R mediate tissue injury over days and weeks, other effects are more immediate. Further, baseline SPL activity in cardiac tissue is relatively low. Thus, experiments were performed to determine whether acute changes in SPL activity occur in response to I/R and, in particular, whether SPL activation might be observed in ventricular tissue at risk of infarction during I/R. Toward that end, the ex vivo Langendorff hanging heart model (see Example 8) was used to induce ischemia, I/R and ischemia preconditioning (IPC) and subsequently assess the effect of these interventions on SPL activity.

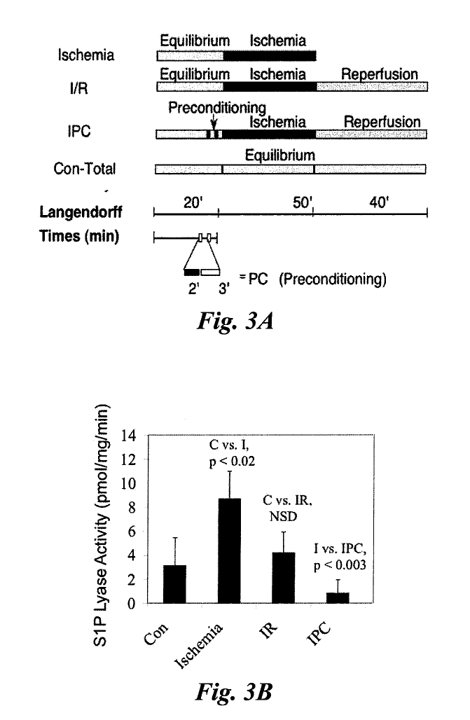

FIG. 3A shows a schematic representation of Langendorff hanging heart protocols used in these experiments. Three or four mice were used for each treatment. The control consisted of 110 minutes of equilibrium. The langendorff method was performed as described previously in Example 8 and as described in Jin Z Q, et al. Circulation 2004; 110(14):1980-9 and Jin Z, et al. Am J Physiol Heart Circ Physiol 2002; 282:H1970-7. SPL activity was determined as described (Bandhuvula P and Saba J. Trends Mol Med 2007; 13:210-217) in LV extracts in control versus ischemia/reperfusion and ischemia versus IPC.

Wild type mice were euthanized by pentobarbital injection, thoracotomy was performed, and hearts were isolated, affixed to the Langendorff apparatus, perfused with physiologic buffer and subjected to one of several different treatment regimens, including perfusion with physiological buffered and oxygenated solution (equilibrium), ischemia (no perfusion) or ischemia followed by reperfusion, as shown in FIG. 3A. Preconditioning, which consisted of two short (2 minute) periods of ischemia during equilibrium periods, was performed prior to I/R. After treatment, hearts were immediately homogenized in SPL assay buffer and frozen. SPL activity assays were then performed on thawed extracts as described (Bandhuvula P, et al. J Biol Chem 2005; 280:33697-33700).

As shown in FIG. 3B, SPL activity was markedly elevated in ischemic tissues to levels approximately three-fold over baseline. Interestingly, SPL activation occurred only in LV but not RV tissues, indicating that the effect is unique to LV myocardium, consistent with their differential sensitivities and molecular properties (Melle C, et al. Int J Mol Med 2006; 18:1207-1215). SPL activation occurred in the absence of increased SPL protein expression as determined by immunoblotting. SPL activity returned to nearly baseline levels in I/R-treated tissues, which, without being bound by theory, could be due to reversibility of the effect or, alternatively, due to cell death. IPC plus ischemia was associated with reduced levels of SPL in comparison to ischemia alone; SPL activity was even lower in IPC plus ischemia-treated tissues than baseline, although the results did not reach statistical significance.

These data demonstrate that SPL is rapidly activated during ischemia, whereas SPL activation is prevented by IPC. These findings suggest that, while SPL gene transcription is induced by hypoxia in cell culture, the enzyme can also be activated through a mechanism most consistent with post-translational events (e.g., phosphorylation, nitrosylation, or other protein modification). Further, SPL activation correlates with injury and decreased SPL activity correlates with protection, raising the possibility that SPL may contribute to ischemic injury by rapidly removing intracellular S1P stores within cells that have lost access to the rich source of SIP in the circulation.

Thus, these data support the concept that inhibiting SPL can be used as a therapy for reducing or preventing cardiac injury.

### Example 13

### Additional Experiments to Determine the Effect of Ischemia/Reperfusion on SPL Expression

Two mouse models of I/R are used for analyzing the interrelationship between ischemia or I/R and SPL expression, activity and function. The Langendorff system, essentially as described in Example 8, is utilized. In the Langendorff system, the animal is deeply anesthetized, the heart is removed through a thoracotomy incision, and the aorta is cannulated above the aortic valves and mounted onto a perfusion system. This allows perfusion of physiological buffer, which is administered retrograde into the aorta, so the aortic valves are closed by the pressure and coronary arteries are filled with perfusion fluid. A balloon placed in the left ventrical (LV) is connected to a pressure transducer, which is connected to a computerized analytical setup, allowing determination of LV function after I/R. Discontinuation of perfusion simulates ischemia, and restoring perfusion mimics recovery. This model is useful for determining intrinsic effects of I/R on cardiac tissue ex vivo.

Hearts are challenged by ischemia and I/R and pretreated or not with IPC. SPL activity and expression is measured by activity assays, immunohistochemistry, quantitative RT-PCR and immunoblotting as described herein (see e.g., Example 14). In addition, a targeted knock-in β-galactosidase reporter system in the null allele of the SPL+/− mouse is used, allowing one additional measure of SPL expression by staining fresh or formalin-fixed tissue slices for β-galactosidase activity.

Without being bound by theory, since SPL is induced through a transcriptional mechanism in response to hypoxia, some effects of I/R on SPL activity may not be manifest until several hours after the ischemic event. To characterize these effects, a surgical model which involves ligation of the left anterior descending artery (LAD), followed by recovery and euthanasia at time points up to 24 h is used. This procedure requires general anesthesia and supportive equipment for artificial ventilation. Thoracotomy is performed, and the coronary artery is ligated with a thread. A small piece of tubing is placed between vessel and thread to minimize injury to the vessel and facilitate reperfusion. Ischemia, induced for 30 minutes, is visible by paleness at the affected site; reperfusion is visible as hyperemia at the site of previous ischemia.

For both ex vivo and in vivo models, heart tissue is analyzed at various time points from within 5 minutes of ischemia (Langendorff), to up to 24 h (surgical ligation) for changes in enzyme activity, gene and protein expression compared to perfused or sham-operated control animal hearts.

### Example 14

### In Vitro Cell-Based Assays to Determine the Effect of Ischemia/Reperfusion on SPL Expression

To dissect which I/R-related conditions affect SPL gene expression, the effects of hypoxia, serum deprivation, glucose depletion and oxidative stress on reporter expression, endogenous SPL expression and activity is measured in relevant transfected cell lines. A DNA fragment containing 1.8 kb of sequences upstream of the ATG start codon of the SPL gene is sufficient to confer hypoxia-induced upregulation to a reporter gene placed under its control. A larger reporter containing 7.8 kb of SPL promoter is also employed. Reporter studies are initially conducted in HEK293 cells due to their high efficiency of transfection, followed by studies conducted in CMG, 3T3 and HV5 cell lines. Reporter constructs are transfected into cells along with either PRL-CMV or pRL-SV40 plasmids to allow for normalization of transfection efficiency. Stable reporter cell lines are generated if necessary. Cells are incubated under the condition of interest and the response is assessed throughout a time course by measuring luciferase activity using Promega's dual luciferase assay system (Promega, Madison, Wis.). Other commercially available luciferase assay systems may also be used. Reporter study results are compared to endogenous SPL protein expression and activity throughout the same time course using standard methods as described herein.

Lac Z reporter analysis in tissues is performed essentially as described (Huang L, et al., Mol Cell Biol 2001; 21:8575-8591). Luciferase reporter plasmids contain 1.8 or 7.8 kb of human Sgpl1(SPL) gene regulatory sequence upstream of the ATG start site cloned into the reporter plasmid pGL3_Basic (Promega, Madison, Wis.). Luciferase assays are performed using commercially available assay systems, such as Steady-Glo Luciferase Assay System (Promega, Madison, Wis.) as described (Oskouian B, et al., J Biol Chem 2005; 280(18):18403-10).

Total RNA is extracted using commercially available RNA isolation kits, such as those available from Qiagen (Valencia, Calif.). RNA quantity and purity are determined spectrophotometrically and integrity is verified using an Agilent 2100 Bioanalyzer. The level of gene expression is assessed by Q-PCR of reverse-transcribed total RNA, using a combination of TaqMan® and SYBR® Green-based chemistries for amplicon detection essentially as described (Oskouian B, et al., Proc Natl Acad Sci USA 2006; 103:17384-17389). Control SYBR® Green primers for six housekeeping genes (β-actin, peptidyl prolyl isomerase B, phosphoglycerate kinase 1, glyceraldehyde-3-phosphate dehydrogenase, β-glucuronidase, transferrin receptor) is used for normalizing tissue SPL gene expression.

### Example 15

### SPL Inhibition for Cardioprotection in Short Term Models of I/R Myocardial Injury

Mice are treated for three days with vehicle or THI administered in the drinking water. The first set of experiments involves euthanizing THI-treated and vehicle-treated mice, isolating hearts, then assessing recovery from I/R using the Langendorff hanging heart ex vivo model of myocardial infarction as described in Example 1 and Example 8. The effect of THI treatment on hemodynamic function, infarction size and pathological changes associated with ischemia/reperfusion in the ex vivo model (i.e. in the absence of circulating blood cells, systemic cytokines, etc.) is assessed. Hemodynamic analysis of left ventricular function includes left ventricular end diastolic pressure (LVEDP), an indicator of heart function and residual fluid volume at the end of diastole, as well as LV developed pressure (peak systolic pressure minus LVEDP), a measure of cardiac function during systole. Infarction size is determined in heart tissue slices by failure to reduce the dye tetrazolium red (TTC), in comparison to healthy tissue which reduces the dye to a visible red color. Apoptosis, necrosis, and other pathological changes in the heart tissue is assessed by morphology in combination with appropriate immunohistochemical stains. Molecular markers of apoptosis, stress, and inflammatory signaling are quantitated in heart tissues using immunoblotting. Effective SPL blockade is verified by SPL activity assays and S1P quantitation in liver and spleen tissue.

The Langendorff model allows assessment of cardiac-intrinsic and very short-term (less than 2 h) effects of I/R. To address the effect of THI pretreatment on cardiac injury manifest from 1-24 hours and potentially involving circulating factors and immune cells in a surviving animal, surgical ligation of the LAD is performed in mice treated with THI or vehicle, as described in Example 13. Mice are euthanized 24 hours after ligation, in order to assess the effects of SPL inhibition on tissue damage including that induced by inflammatory changes and apoptosis which may be evident only at later time points. Endpoints are essentially the same as described for the Langendorff model, except that pathological analysis is performed in one cohort of animals, and hemodynamic studies are conducted ex vivo using the Langendorff model in a second cohort of animals.

The Langendorff isolated perfused heart preparation, I/R and IPC protocols, hemodynamics, and infarct size determination by TTC are performed essentially as described (Jin Z, et al., Am J Physiol Heart Circ Physiol 2002; 282:H1970-7). Surgical ligation of the LAD is performed as described for rats and mice (Yeh C-C, et al., Circulation 2006; 114:(Suppl): II-138; Zhu B, et al., Cardiovasc Drugs Ther 2004; 18:421-431). These methods are also described in detail in Chimenti et al. (Chimenti S, et al., Methods Mol Med 2004; 98:217-26) and more recently by Klocke et al. (Klocke R, et al., Cardiovasc Res 2007; 74(1):29-38). Briefly, thirty minutes of transient myocardial ischemia followed by 24 h reperfusion is performed according to an IACUC-approved protocol on barbiturate-anaesthetized mice. Thoracotomy and ligation of the LAD coronary artery are performed at the level of the left atrium with a silk 7.0 suture tied over PE10 tubing. After chest closure, animals are weaned from the ventilator and observed until fully recovered and active. After 24 h, animals are euthanized and hearts excised for pathological analysis or hemodynamic analysis. THI will be given at a dose of 50 mg/L ad libitum for three days (Phillips J A and Paine A J. Xenobiotica 1990; 20(6):555-62).

Area of infarction and localization of lesions is determined. Areas of necrosis and apoptosis and endothelial vs. cardiomyocyte cell death are assessed. TUNEL and processed caspase-3 assays are performed on formalin-fixed paraffin-embedded sections using the ApoTag kit (Oncor, Gaithersburg, Md.) and the Cleaved Caspase-3 Detection Kit (Cell Signaling, Beverly, Mass.). The number of TUNEL or caspase-3 positive cells per 1000 total cells are counted in each of 3 to 5 random fields. Polymorphonuclear cells (PMNs) are identified using MCA771G antibody (Serotec, Raleigh, N.C.) (Theilmeier G, et al. Circulation 2006; 114(13):1403-9). Endothelium is identified using rat monoclonal anti-PECAM-1 (Chemicon/Millipore, Billerica, Mass.) as described (Kohno M, et al. Mol Cell Biol 2006; 26:7211-7223).

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for reducing or preventing cardiac injury in a subject known to have, or to be at risk for sustaining, cardiac injury, comprising administering to the subject an agent that inhibits sphingosine-1-phosphate lyase (SPL) activity and thereby reducing or preventing cardiac injury in the subject.

2. A method for reducing or preventing stroke injury in a subject known to have, or to be at risk for sustaining, stroke injury, comprising administering to the subject an agent that inhibits sphingosine-1-phosphate lyase (SPL) activity and thereby reducing or preventing stroke injury in the subject.

3. A method for preventing or reducing tissue injury due to organ transplantation in a subject, comprising administering to the subject an agent that inhibits sphingosine-1-phosphate lyase (SPL) activity and thereby preventing or reducing tissue injury due to organ transplantation.

4. The method of any one of claims 1-3 wherein the agent that inhibits SPL activity comprises 2-acetyl-4-tetrahydroxybutylimidazole (THI).

5. The method of claim 4 further comprising administering to the subject at least one of a beta-blocker and an antioxidant.

6. The method of claim 5 wherein the beta-blocker is selected from the group consisting of acebutolol, betaxolol, carteolol, labetalol, metoprolol and propranolol and the antioxidant is selected from the group consisting of ascorbic acid and sodium bisulfite.

7. The method of any one of claims 1-3 wherein the agent that inhibits SPL activity comprises at least one antibody that specifically binds to a human SPL polypeptide which comprises the amino acid sequence as set forth in any one of SEQ ID NOS:8, 10 and 18.

8. The method of claim 7 wherein the antibody is selected from the group consisting of a polyclonal antibody, a monoclonal antibody, a chimeric antibody, a humanized antibody, a Fab fragment, a Fab′ fragment, a (Fab′)₂ fragment, an Fd fragment, an Fv fragment, an scFv, a dAb and a diabody.

9. The method of any one of claims 1-3 wherein the agent that inhibits SPL activity comprises an antisense nucleic acid that specifically hybridizes to a human SPL encoding polynucleotide which comprises the nucleotide sequence set forth in any one of SEQ ID NOS:7, 19 and 17.

10. The method of any one of claims 1-3 wherein the agent that inhibits SPL activity comprises an RNAi molecule that interferes with expression of a SPL polypeptide having SPL activity, wherein the SPL polypeptide having SPL activity is selected from (i) a polypeptide that comprises the amino acid sequence as set forth in any one of SEQ ID NOS:8, 10 and 18, (ii) a polypeptide that is encoded by the nucleotide sequence set forth in any one of SEQ ID NOS:7, 19 and 17, and (iii) a polypeptide that is encoded by a polynucleotide that is capable of hybridizing under moderately stringent conditions to a nucleic acid having the nucleotide sequence set forth in any one of SEQ ID NOS:7, 19 and 17, or a complementary sequence thereto.

11. The method of any one of claims 1-3 wherein the agent that inhibits SPL activity comprises a ribozyme that interferes with expression of a SPL polypeptide having SPL activity, wherein the SPL polypeptide having SPL activity is selected from (i) a polypeptide that comprises the amino acid sequence as set forth in any one of SEQ ID NOS:8, 10 and 18, (ii) a polypeptide that is encoded by the nucleotide sequence set forth in any one of SEQ ID NOS:7, 19 and 17, and (iii) a polypeptide that is encoded by a polynucleotide that is capable of hybridizing under moderately stringent conditions to a nucleic acid having the nucleotide sequence set forth in any one of SEQ ID NOS:7, 19 and 17, or a complementary sequence thereto.

12. The method of claim 1, wherein the cardiac injury comprises acute ischemia/reperfusion injury.

13. The method of claim 12, wherein the acute ischemia/reperfusion injury is due to one or more events selected from the group consisting of coronary obstruction, cardiac percutaneous intervention, coronary artery bypass surgery, cardiopulmonary bypass and non-cardiac surgery.

14. The method of claim 1, wherein the cardiac injury comprises an injury resulting from surgical repair of congenital heart disease.

15. The method of claim 1, wherein the cardiac injury comprises injury resulting from closure of septal defects by percutaneous means.

16. The method of claim 1, wherein the cardiac injury comprises injury resulting from percutaneous mitral valve repair or mitral valvulotomy.

17. The method of claim 1, wherein the cardiac injury comprises injury resulting from hypoxia.

18. The method of claim 1 wherein the cardiac injury comprises injury resulting from hypoxia with reperfusion.

19. The method of claim 1 wherein the cardiac injury comprises injury resulting from hypoxia with reoxygenation.

20. The method of claim 1, wherein the cardiac injury comprises injury resulting from myocardial infarction.

21. The method of claim 1, wherein the cardiac injury comprises injury resulting from acute congestive heart failure.

22. The method of claim 1, wherein the cardiac injury comprises injury resulting from chronic congestive heart failure.

23. The method of claim 1, wherein the cardiac injury comprises injury resulting from myocarditis.

24. The method of claim 1, wherein the cardiac injury comprises injury resulting from cardiotoxicity of a drug.

25. The method of claim 24 wherein the drug is selected from the group consisting of anti-Her2 antibodies and anthracyclines.

26. The method of claim 1 wherein the cardiac injury comprises injury resulting from cardiotoxicity of radiation treatment.

27. The method of claim 1, wherein the cardiac injury comprises injury resulting from heart transplantation.

28. The method of claim 1, wherein the cardiac injury comprises injury resulting from iron overload.

29. The method of claim 2, wherein the stroke injury comprises acute ischemia/reperfusion injury.

30. The method of claim 2, wherein the stroke injury comprises injury resulting from hypoxia.

31. The method of claim 2 wherein the stroke injury comprises injury resulting from hypoxia with reperfusion.

32. The method of claim 2 wherein the stroke injury comprises injury resulting from hypoxia with reoxygenation.

33. The method of claim 2, wherein the stroke injury comprises injury accompanying toxic dementia.

34. The method of claim 2, wherein the stroke injury comprises injury resulting from vascular dementia.

35. The method of claim 2, wherein the stroke injury comprises injury accompanying Alzheimer's disease.

36. The method of claim 2, wherein the stroke injury comprises injury due to neurotoxicity.

37. A method for reducing or preventing ischemia/reperfusion injury in a tissue in a mammal comprising, administering to said mammal an agent that inhibits SPL activity.
